(19) United States
(12) Patent Application Publication
(10) Pub. No.: US 2015/0164467 A1
(43) Pub. Date: Jun. 18, 2015

(54) WAVEFORM TRACKING DEVICE, ULTRASOUND DIAGNOSTIC DEVICE, AND WAVEFORM TRACKING METHOD

(71) Applicant: FURONO ELECTRIC CO., LTD., Nishinormiya City (JP)

(72) Inventors: Ryoichi Suetoshi, Nishinomiya City (JP); Dorian Cretin, Nishinomiya City (JP)

(21) Appl. No.: 14/415,341

(22) PCT Filed: Jun. 11, 2013
(86) PCT No.: PCT/JP2013/066049

§ 371 Date: Jan. 16, 2015

(30) Foreign Application Priority Data
Jul. 18, 2012 (JP) 2012-159557

(51) Int. Cl. A61B 8/08; A61B 8/14; A61B 8/00; A61B 5/00

(52) U.S. Cl. CPC A61B 8/0875; A61B 5/7264; A61B 8/145; A61B 8/5207; A61B 8/5223; A61B 5/7275; A61B 8/4483

(57) ABSTRACT

A bone strength diagnostic device comprises oscillators, a determination index calculator, a tracking start point setting component, and a tracking execution component. A plurality of the oscillators are disposed in a line to acquire echo signals of a transmitted ultrasound beam. The determination index calculator calculates a determination index expressing a probability that the echo signal is a reflected wave from the surface of a cortical bone. The tracking start point setting component sets a tracking start point at a signal waveform timing corresponding to the echo signal for which the determination index satisfies a specific condition and that is received by one of the plurality of oscillators. The tracking execution component successively sets tracking points for the echo signals acquired from other oscillators using the tracking start point as an origin.

### CROSS REFERENCE TO RELATED APPLICATIONS

This application is a U.S. National stage of International Application No. PCT/JP2013/066049 filed on Jun. 11, 2013. This application claims priority to Japanese Patent Application No. 2012-159557 filed on Jul. 18, 2012. The entire disclosure of Japanese Patent Application No. 2012-159557 is hereby incorporated herein by reference.

### BACKGROUND

1. Field of the Invention

The present invention relates mainly to a waveform tracking device that directs an ultrasound beam at a measurement object, acquires echo signals, and tracks the echo signals by utilizing the fact that there is continuity in the shape of the measurement object.

2. Background Information

A diagnostic device that makes use of ultrasound performs echo tracking processing to track a measurement object (such as blood vessel walls) on the basis of echo signals obtained from the ultrasound beam. Measuring a shape or position of a measurement object based on the result of echo tracking processing is conventional technology. Japanese Patent No. 4,667,177 (Patent Literature 1) discloses an ultrasound diagnostic device of this type.

The ultrasound diagnostic device disclosed in Patent Literature 1 forms a plurality of ultrasound beams, and acquires an echo signal for each ultrasound beam. When a tracking point is manually set by the technician for one ultrasound beam that serves as a reference, the tracking points for the other ultrasound beams are automatically set by the device.

A tracking point is set as follows in Patent Literature 1. The ultrasound diagnostic device specifies a partial echo signal string of a related beam corresponding to a partial echo signal string of the reference beam on the basis of the correlation (similarity) between partial echo signal strings, for the reference beam and a related beam out of the plurality of ultrasound beams. After this, the ultrasound diagnostic device uses this result to detect the position of the reference beam on the related beam corresponding to the tracking point. The tracking point of the related beam is set to this detected position.

### SUMMARY

In the configuration of Patent Literature 1, if the tracking point that serves as the origin for tracking (hereinafter also referred to as the tracking start point) is set incorrectly, there is the risk that an echo of something other than the measurement object will end up being tracked, so it is extremely important to set it correct. However, in setting the tracking start point in the configuration of Patent Literature 1, the tracking point cannot be found by calculating correlated values, and instead the technician has to perform the operation manually. Therefore, not only does the measurement entail a great deal of time and work, but to a certain extent the measurement precision ends up being dependent on the skill of the technician, making it difficult to obtain consistent measurement results. Furthermore, since the tracking in Patent Literature 1 is done by computing the correlation (similarity), the processing burden tends to be greater. Thus, improvement is needed in terms of lowering the cost of the device or shorting the time tracking takes.

The present invention was conceived in light of the above situation, and it is an object thereof to provide a waveform tracking device with which an accurate tracking start point can be reliably set, and the speed of tracking processing can be easily increased.

The problem to be solved by the present invention is as given above, and next the means for solving this problem, and the effect thereof will be discussed.

The waveform tracking device of the present invention comprises wave receivers, a determination index calculator, a tracking start point setting component, and a tracking execution component. A plurality of the wave receivers are disposed in a line to acquire echo signals of a transmitted ultrasound beam. The determination index calculator is configured to calculate a determination index expressing a probability that the echo signal is a reflected wave from a measurement object. The tracking start point setting component is configured to set a tracking start point for an echo signal for which the determination index satisfies a specific condition, and that has been received by some of the plurality of wave receivers. The tracking execution component is configured to successively set tracking points for the echo signals acquired from other wave receivers using the tracking start point as an origin.

No tracking start point is set when the determination index does not satisfy the specific condition. Therefore, erroneous setting of the tracking start point can be prevented. As a result, this prevents mistakes in which an unintended echo is tracked, affording more robust waveform tracking.

With this waveform tracking device, it is preferable if, when the determination index satisfies a specific condition for an echo signal with a maximum peak out of the echo signals acquired by the plurality of wave receivers, the tracking start point setting component sets the tracking start point at a signal waveform timing corresponding to the echo signal. Since an accurate tracking start point can be set automatically, a waveform tracking device that is simpler to operate can be provided. Since the setting of the tracking start point does not rely on the skill of the device user, tracking accuracy can be consistently improved.

It is preferable if the determination index is calculated so that the probability that the echo signal is a reflected wave from a measurement object becomes higher in at least one of the following cases: a peak expressed by a waveform of the echo signal is strong, the peak is sharp, and if there is little distortion in the waveform. Since the determination index can be calculated by means of a rational criterion, the tracking start point can be found more accurately.

It is preferable if at least a value that evaluates a size relation between a plurality of peaks had by the echo signals is used as the determination index. Evaluating the size relation between the plurality of peaks allows the probability that an echo signal is a reflected wave from the measurement object to be rationally determined without imposing an excessively heavy processing load.

It is preferable if at least a value that evaluates a relation between a size of a maximum peak that is a largest peak out of a plurality of peaks had by the echo signals, and a size of an adjacent peak adjacent to this maximum peak is used as the determination index. Since the sharpness of the maximum peak can be properly evaluated, whether or not an echo signal can be trusted to be a reflected wave from the measurement object can be accurately determined.

It is preferable if when a largest peak out of a plurality of peaks had by the echo signals is a maximum peak, a peak adjacent to the maximum peak on one side is one side adjacent peak, and a peak adjacent to the maximum peak on the other side is the other side peak, at least a value that evaluates a relation between a size of the maximum peak and a difference between a size of the one side adjacent peak and a size size of the other side adjacent peak is used as the determination index. Since the extent of distortion of the echo signal can be properly evaluated, it can be accurately determined whether or not the echo signal can be trusted to be a reflected wave from the measurement object.

It is preferable if the tracking start point setting component sets the tracking start point at the signal waveform timing corresponding to the echo signal acquired by a wave receiver that is not at an end of the line of the wave receivers. Even if the measurement object has a curved shape, tracking can be reliably carried out by starting the tracking from the middle.

The tracking execution component sets a specific window period in a time axis direction centered on a point corresponding to the tracking start point or the tracking point from a signal waveform received by a wave receiver adjacent to the wave receiver that acquires an echo signal to which the tracking start point or the tracking point belongs (called an “adjacent signal waveform”). The tracking execution component takes out a partial waveform within the window period, and checks whether or not an echo signal that satisfies the determination index exists. If exists, then the tracking point is set at a signal waveform timing corresponding to the echo signal. If the determination index does not satisfy the specific condition, no tracking point is set. Since no tracking point is set, this prevents the tracking from going too far to an echo that is not attributable to the measurement object. As a result, more robust waveform tracking can be performed.

If no echo signal that satisfies the determination index exists in the partial waveform, the tracking execution component takes out a partial waveform by setting a specific second window period in a time axis direction centered on a point corresponding to the tracking start point or the tracking point from the adjacent signal waveform. The tracking point is set at a signal waveform timing corresponding to the maximum peak in this partial waveform. The second window period is set to be a shorter time than the window period. Even if the determination index does not satisfy the specific condition, as long as a large peak is found within a short time period, the tracking point will be set at a timing corresponding to that peak. Therefore, there will be a better balance between continuity and accuracy of waveform tracking.

It is preferable if the tracking execution component skips setting of a tracking point with respect to the adjacent signal waveform if the maximum peak in the partial waveform taken out by setting the second window period is smaller than a specific size. If the echo is weak, the setting of the tracking point will be skipped rather than being forced, so tracking mistakes can be prevented more reliably.

A waveform tracking device comprises a signal image generator and a predicted region image generator. The signal image generator is configured to divide the signal waveforms acquired by the plurality of wave receivers into a plurality of parts in the time axis direction, respectively, to digitize them, and configured to arrange them in a matrix to obtain a signal image. The predicted region image generator is configured to subject the signal image to image processing to obtain a predicted region image indicating a region where there is a high probability that an echo signal corresponding to a shape of the measurement object is included. The tracking start point setting component sets the tracking start point only for an echo signal of a time period and the wave receiver corresponding to the region indicated by the predicted region image. Since the inclination of the signal waveforms acquired by the plurality of wave receivers can be considered in the form of an image, erroneous setting of the tracking start point can be more reliably prevented.

It is preferable if the tracking execution component sets the tracking point only for an echo signal of the time period and the wave receiver corresponding to the region indicated by the predicted region image. Since the inclination of the signal waveforms acquired by the plurality of wave receivers can be considered in the form of an image, tracking mistakes can be more reliably prevented.

With the ultrasound diagnostic device of the present invention, accurate diagnosis of the interior of a subject is possible by sending ultrasound beams to the subject and receiving echo signals thereof, using the same principle as discussed above. For example, this can be applied to an osteochondral diagnostic device for diagnosing the osteochondral state of a person.

There is also provided the following waveform tracking method that is used in a waveform tracking device having a plurality of wave receivers disposed in a line to acquire echo signals of a transmitted ultrasound beam. The waveform tracking method comprises a determination index calculation step, a tracking start point setting step, and a tracking execution step. The determination index calculation step involves calculating a determination index that expresses a probability that the echo signal is a reflected wave from a measurement object. The tracking start point setting step involves setting a tracking start point for the echo signal for which the determination index satisfies a specific condition and that is received by a part of the plurality of wave receivers. The tracking execution step involves successively setting tracking points for the echo signals acquired from other wave receivers using the tracking start point as an origin. If the determination index does not satisfy the specific condition, no tracking point is set, and this prevents the erroneous setting of the tracking start point. As a result, this prevents mistakes in which an unintended echo is tracked, affording more robust waveform tracking.

### BRIEF DESCRIPTION OF THE DRAWINGS

FIG. 1 is a simplified cross section and a function block diagram of a bone strength diagnostic device pertaining to a first embodiment of the present invention.

FIG. 2 is a concept diagram illustrating a plurality of propagation paths of ultrasonic waves.

FIG. 3 is a graph of examples of waveform signals outputted by various oscillators.

FIG. 4 is a flowchart of a sonic speed measurement method in this embodiment.

FIG. 5A shows how planar waves are sent out by an oscillator array, and FIG. 5B shows how the planar waves sent out by the oscillator array are reflected at the front or back of cortical bone.

FIG. 6 is a graph of an example of a waveform when an oscillator has received an echo with a sharp peak.

FIG. 7 is a graph of an example of a waveform when an oscillator has received an echo that has undergone positive/negative inversion.

FIG. 8 is a graph of an example of a waveform when an oscillator has received an echo that has spread out somewhat in the time axis direction.

FIG. 9 is a graph of how a pair of tracking start points is set among signal waveforms received by various oscillators.

FIG. 10 is a graph illustrating the method for newly setting a tracking point on the basis of the signal waveform of the oscillator being tracked.

FIG. 11 is a graph of the results obtained by tracking the signal waveforms received by the oscillators.

FIG. 12A is a simplified detail view of the area near an oscillator group receiving surface reflected waves, and FIG. 12B is a simplified diagram illustrating the difference in the propagation paths of surface reflected waves reaching two oscillators that make up an oscillator group.

FIG. 13 is a function block diagram of the configuration of a sonic speed sensor.

FIG. 14 is a simplified diagram illustrating a method for calculating the propagation path of a surface refracted wave.

FIG. 15 is a graph of a t-x curve found by a postulated propagation time calculator.

FIG. 16 shows how a window function is multiplied by the waveform signals of various oscillators.

FIG. 17 shows how the waveform signals of various oscillators are shifted.

FIG. 18 shows an integrated waveform found by a waveform integrator.

FIG. 19 shows how the t-X curve is plotted by varying a sonic speed postulated value.

FIG. 20 is a graph of an example of the results of plotting the amplitude of an envelope while varying the sonic speed postulated value.

FIG. 21 is a function block diagram showing the configuration of a waveform tracker in the bone strength diagnostic device in a second embodiment.

FIG. 22 is a diagram of a signal image produced on the basis of the signal waveforms received by the various oscillators.

FIG. 23 is a diagram of a predicted region image produced from the signal image.

### DETAILED DESCRIPTION OF EMBODIMENTS

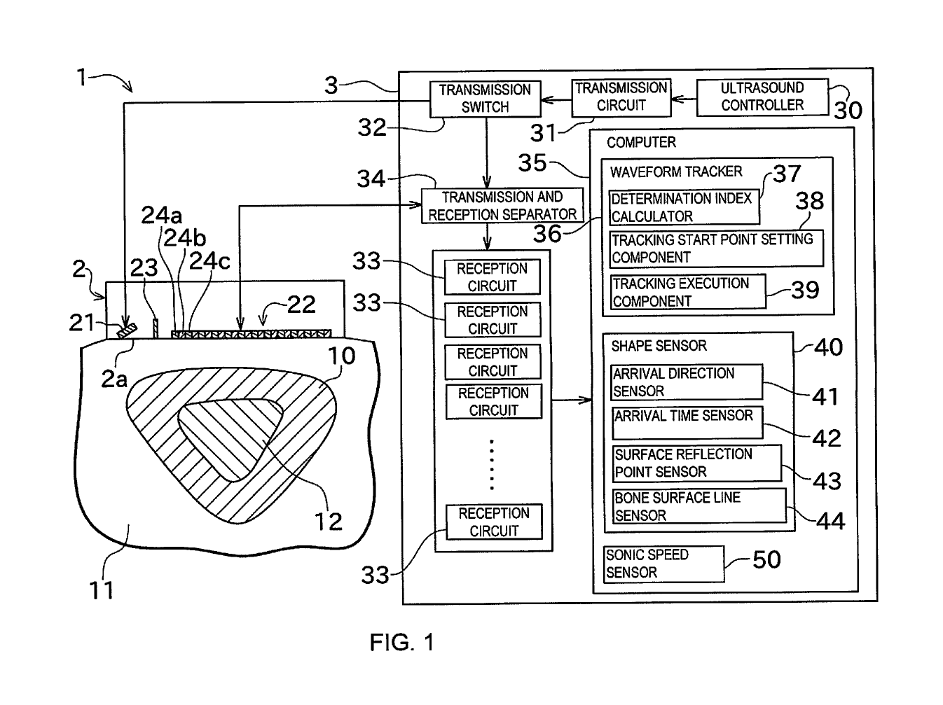

Embodiments of the invention will now be described through reference to the drawings. FIG. 1 is a simplified cross section and a function block diagram of a bone strength diagnostic device 1 pertaining to an embodiment of the present invention.

The bone strength diagnostic device (waveform tracking device, shape sensing device, sonic speed measurement device) 1 diagnoses, for example, the bone strength of cortical bone with a long tubular shape, such as a tibia (what is diagnosed is not, however, limited to this). More specifically, bone is generally made up of cortical bone 10 and spongy cancellous bone 12 that is present on the inside of the cortical bone 10. The outside of the cortical bone 10 is covered by soft tissue 11, such as muscle or fat. The bone strength diagnostic device 1 in this embodiment is configured so that ultrasonic waves are emitted from the outside of the soft tissue 11 toward the cortical bone 10, and the speed of sound through the cortical bone 10 (bone sonic speed) is measured. Therefore, in this embodiment, the cortical bone 10 corresponds to the measurement object.

The left side in FIG. 1 is a cross section in which a portion of a person's tibia, taken along a plane perpendicular to the lengthwise direction of the bone. As shown in FIG. 1, the contour shape of the surface of the cortical bone 10 is a gentle curve that bulges out in the radial direction (the direction perpendicular to the lengthwise direction of the bone. In the following description, a long tubular bone will be thought of as a circular column, and the direction in which the ultrasonic waves propagate along the surface of the cortical bone 10 in this cross section will sometimes be called the peripheral direction. Although not depicted in the drawings, in a cross section taken along a plane parallel to the lengthwise direction of the bone, the surface contour of the cortical bone 10 is substantially linear. The bone strength diagnostic device 1 in this embodiment is configured to be able to accurately measure the speed of sound even when the cross sectional contour of the measurement object is curved, as when measuring the speed of sound in the peripheral direction of a bone.

The configuration of the bone strength diagnostic device 1 will now be described. As shown in FIG. 1, the bone strength diagnostic device 1 is made up of an ultrasonic wave transceiver 2 and a device main body 3.

The ultrasonic wave transceiver 2 sends and receives ultrasonic waves. This ultrasonic wave transceiver 2 comprises a contact face 2a that comes into contact with the surface of the soft tissue 11 at the measurement site, a dedicated wave transmission oscillator 21, an oscillator array 22, and a sound insulator 23. The oscillator array 22 is composed of a plurality of oscillators 24 that are disposed in a single line. The dedicated wave transmission oscillator 21 and the oscillators sound insulator 23 are disposed in line along the layout direction of the oscillator array 22. This embodiment features oscillators whose surface vibrates and emits ultrasonic waves when an electrical signal is applied, and which produce and output electrical signals upon receiving ultrasonic waves on their surface.

The dedicated wave transmission oscillator (wave transmitter) 21 is installed so that is surface is inclined with respect to the contact face 2a, and is configured to be able to send ultrasonic waves obliquely from the contact face 2a. The dedicated wave transmission oscillator 21 used here is one with weak directionality of the emitted ultrasonic waves (a wide angle range of the ultrasonic waves).

The oscillator array 22 comprises a plurality of oscillators (wave receivers) 24. In the following description, when it is necessary to distinguish between the plurality of oscillators 24, they will be given lower-case letter suffixes, starting from the side closest to the dedicated wave transmission oscillator 21, and expressed as the oscillator 24a, the oscillator 24b, the oscillator 24c, and so forth. These oscillators 24 are disposed in a single line, equidistantly spaced, so as to be parallel to the contact face 2a. Also, the oscillators 24 are configured to be capable of sending and receiving ultrasonic waves.

The sound insulator 23 is formed in a flat shape, and is disposed between the dedicated wave transmission oscillator 21 and the oscillator array 22. The sound insulator 23 prevents the ultrasonic waves sent out from the dedicated wave transmission oscillator 21 from propagating through the ultrasonic wave transceiver 2 and directly reaching the oscillator array 22. The material of the sound insulator 23 can be, for example, a material having a sound absorption effect, such as cork, synthetic rubber, or a porous substance (such as foamed resin).

When the ultrasonic wave transceiver 2 is actually used to send and receive ultrasonic waves, an ultrasound jelly is applied to the skin surface at the measurement site (that is, the outer surface of the soft tissue 11), and the contact face 2a is brought into contact with this skin surface. Ultrasonic waves are sent and received by the dedicated wave transmission oscillator 21 and the oscillator array 22. Consequently, the ultrasonic waves go through the soft tissue 11 and hit the cortical bone 10, which is the measurement object. The ultrasonic waves that come back from the cortical bone 10 are received by the oscillator array 22. The ultrasound jelly prevents gaps from forming between the soft tissue 11 and the contact face 2a, and also matches the acoustic impedance between the contact face 2a and the soft tissue 11, which minimizes reflection at the surface of the soft tissue 11 of the ultrasonic waves sent from the dedicated wave transmission oscillator 21 or the oscillator array 22.

Next, the device main body 3 will be described. The device main body 3 is connected by cable to the ultrasonic wave transceiver 2, and is configured to allow signals to be sent to and received from the ultrasonic wave transceiver 2. More specifically, this device main body 3 comprises an ultrasound controller 30, a transmission circuit 31, a transmission switch 32, a plurality of reception circuits 33, a transmission and reception separator 34, and a computer 35.

The transmission circuit 31 is configured so as to produce electrical pulse signals for generating ultrasonic waves by vibrating the dedicated wave transmission oscillator 21 or the oscillator array 22, and to send these electrical pulse signals to the transmission switch 32. The center frequency of the electrical pulse vibration is about 1 to 10 MHz, for example. A chirp signal may be used instead of an electrical pulse signal, for example.

The transmission circuit 31 is configured so that when ultrasonic waves are generated by the oscillator array 22, electrical pulse signals can be produced at the desired timing for each of the oscillators 24. The ultrasound controller 30 is connected to the transmission circuit 31, and is configured to send the transmission circuit 31 control signals for sending out ultrasonic waves from the oscillators 24. It is possible for the control to be such that the ultrasonic waves are sent all at once from the oscillators 24, or at individual timing.

The transmission switch 32 is configured so that electrical pulse signals sent from the transmission circuit 31 can be switched to be sent to either the dedicated wave transmission oscillator 21 or the oscillator array 22. Specifically, the transmission switch 32 selects which oscillators will send out ultrasonic waves.

The reception circuits 33 are connected corresponding to the oscillators 24 that make up the oscillator array 22. The reception circuits 33 are configured to receive electrical signals outputted when the oscillators 24 receive ultrasonic waves. The reception circuits 33 are configured to produce digital signals that have undergone amplification, filtering, digital conversion, and other such processing to the electrical signals, and send these to the computer 35. The signals directly outputted from the oscillator array 22 are analog waveform signals, and the signals transmitted to the computer 35 are digital waveform signals that have undergone signal processing. In the following description, no distinction will be made between these forms, and they will be referred to simply as “waveform signals.”

The transmission and reception separator 34 is connected between the oscillator array 22 and the transmission circuit 31 and reception circuits 33. The transmission and reception separator 34 prevents the electrical signals (electrical pulse signals) sent from the transmission circuit 31 to the oscillator array 22 from flowing directly to the reception circuits 33. This is also for preventing the electrical signals sent from the oscillator array 22 to the reception circuits 33 from flowing to the transmission circuit 31 side.

Next, how the ultrasonic waves are transmitted by the dedicated wave transmission oscillator 21 will be described. When ultrasonic waves are transmitted by the dedicated wave transmission oscillator 21, the transmission switch 32 causes the dedicated wave transmission oscillator 21 to determine which oscillators will send out ultrasonic waves. When a pulse signal is sent from the transmission circuit 31 to the dedicated wave transmission oscillator 21, the dedicated wave transmission oscillator 21 sends an ultrasonic wave that has undergone pulse modulation in a direction that is oblique to the cortical bone 10.

The ultrasonic waves sent out from the dedicated wave transmission oscillator 21 are received by the oscillator array 22, via a plurality of propagation paths. When the oscillator array 22 receives the ultrasonic waves, waveform signals are sent from the oscillators 24 to the computer 35.

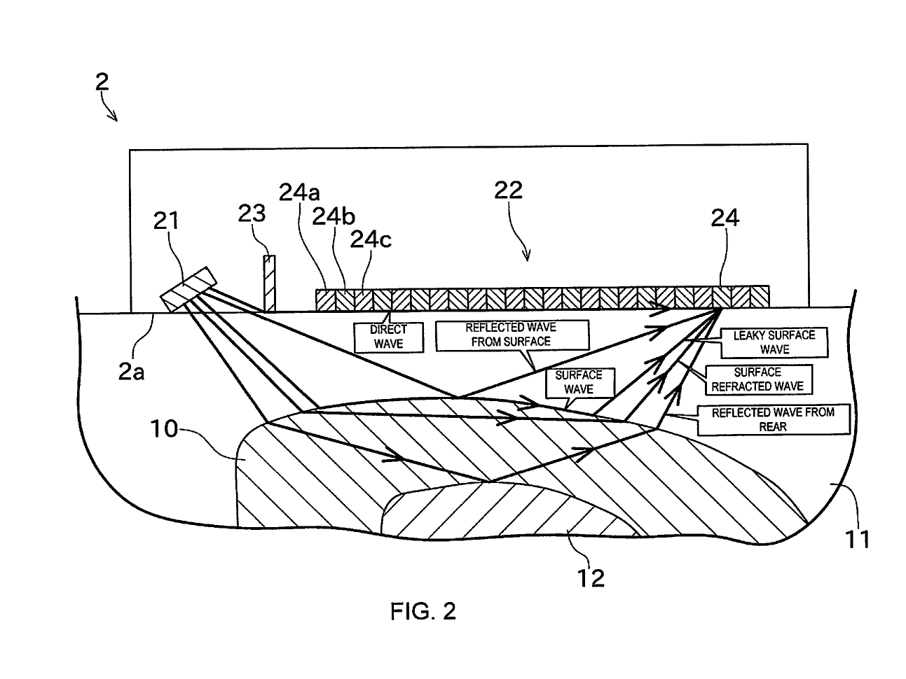

The paths over which the ultrasonic waves sent from the dedicated wave transmission oscillator 21 propagate will be described through reference to FIG. 2. FIG. 2 is a concept diagram of a plurality of propagation paths of ultrasonic waves that reach a certain oscillator 24. Only an example of ultrasonic waves reaching a single oscillator 24 is shown in FIG. 2, but in actual practice ultrasonic waves can reach all of the oscillators 24 along a plurality of paths.

As shown in FIG. 2, direct waves directly reach the oscillator 24 when the ultrasonic waves sent from the dedicated wave transmission oscillator 21 propagate along the surface of the soft tissue 11. Reflected waves from the surface reach the oscillator 24 when the ultrasonic waves sent from the dedicated wave transmission oscillator 21 propagate through the soft tissue 11 and are reflected at the surface of the cortical bone 10 (the boundary between the soft tissue 11 and the cortical bone 10). The reflected waves from the back reach the oscillator 24 when the ultrasonic waves that have propagated through the soft tissue 11 are incident inside the cortical bone 10, propagate through the cortical bone 10, are reflected by the back of the cortical bone 10 (the boundary between the cortical bone 10 and the cancellous bone 12), and are then emitted again into the soft tissue 11.

It is also possible that the ultrasonic waves sent from the dedicated wave transmission oscillator 21 will propagate through the soft tissue 11, hit the cortical bone 10, propagate near the surface of the cortical bone 10, and then be emitted again into the soft tissue 11 before reaching the oscillator 24. In this Specification, an ultrasonic wave that is received via a propagation path such as this will be called a surface propagation wave. Surface propagation waves include two propagation paths: leaky surface waves and surface refracted waves.

When the ultrasonic waves hit the surface of the cortical bone 10 at a critical angle, surface waves are generated at the surface of the cortical bone 10. These surface waves emit leaky waves in a specific direction (the direction where the emission angle becomes the critical angle) on the soft tissue 11 side, while propagating along the surface of the cortical bone 10. The leaky waves received by the oscillators 24 here are called “leaky surface waves.” Meanwhile, when the ultrasonic waves hit the surface of the cortical bone 10 at an angle that is less than the critical angle, they are refracted at the cortical bone 10 surface. If the ultrasonic waves here are incident at an angle that is close to the critical angle, they propagate within the cortical bone 10 and near the surface of the cortical bone 10, after which they are refracted at the cortical bone 10 surface on the oscillator array 22 side, and are radiated into the soft tissue 11. The ultrasonic waves received here shall be called “surface refracted waves” in this Specification. These surface refracted waves are generated only when the cross sectional contour shape of the cortical bone 10 is curved.

Depending on the shape of the bone, the position of the oscillators 24 that receive the waves, the angle of the ultrasonic waves sent from the dedicated wave transmission oscillator 21, and other such conditions, there may be situations when some of the plurality of types of ultrasonic waves mentioned above are not generated, or even if they are generated, they may not be received by the oscillators 24. In this embodiment, however, since the dedicated wave transmission oscillator 21 with broad directionality is used as mentioned above, the ultrasonic waves can hit the cortical bone 10 at the critical angle or at an angle that is close to the critical angle. Leaky surface waves or surface refracted waves can be reliably generated, and these leaky surface waves or surface refracted waves can reach at least one of the plurality of oscillators 24.

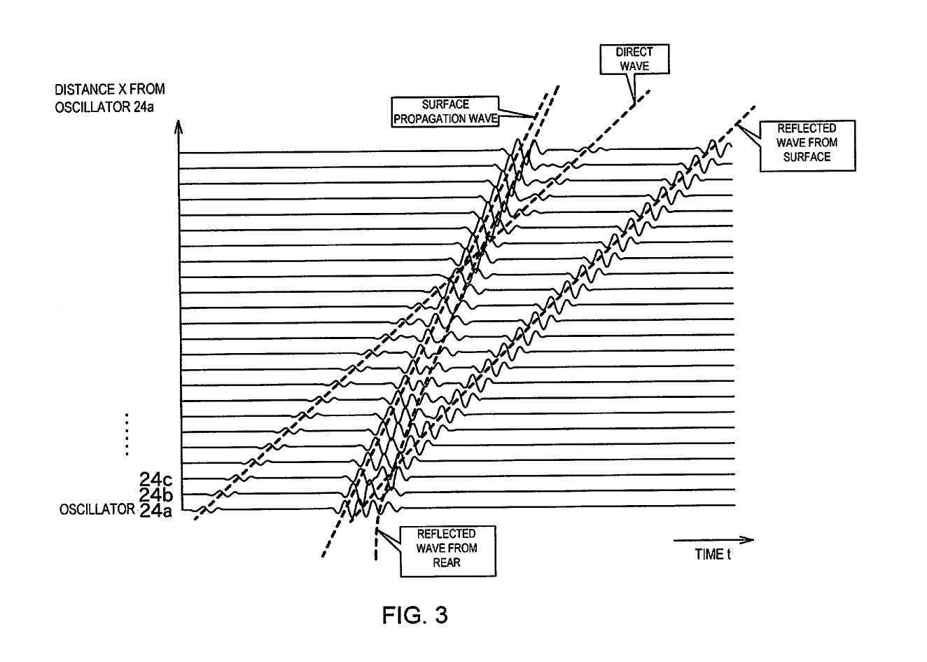

Next, the signal waveforms generated by the oscillators 24 will be described through reference to FIG. 3. FIG. 3 is a graph of the waveforms of waveform signals outputted by various oscillators upon receiving ultrasonic waves, after the dedicated wave transmission oscillator 21 has sent out the ultrasonic waves. In the waveforms of the oscillators 24, the horizontal axis t is time, and the vertical axis of the waveform signals is the amplitude of these waveform signals. The x axis in the graph shows the distance from an oscillator 24a, which is the oscillator closest to the dedicated wave transmission oscillator 21, to the other oscillators 24b, 24c, etc.

As the receiving oscillator 24 moves away from the dedicated wave transmission oscillator 21, the amplitude of the waveform signal decreases. The curve of the waveforms shown in FIG. 3 is adjusted by multiplying each waveform signal by a suitable gain so that the various waveform signals will have approximately the same amplitude.

As shown in FIG. 3, the waveform signal of each oscillator 24 includes a plurality of peaks. In FIG. 3, the positions of the peaks included in the waveform signal of each oscillator 24 are connected by a dotted line to indicate whether those peaks are produced by direct waves, reflected waves from the front, reflected waves from the back, or surface propagation waves. In this graph, it is difficult to distinguish between leaky surface waves and surface refracted waves, so the two are collectively shown as surface propagation waves.

As can be seen from the graph in FIG. 3, direct waves may reach the oscillators 24 before the surface propagation waves, or may reach them after. Reflected waves from the back and reflected waves from the front may sometimes reach the oscillators 24 at substantially the same time as the surface propagation waves. In this case, the peaks will overlap each other, making them hard to tell apart. If such peaks are included in a waveform signal, it will be difficult to determine the path over which an ultrasonic wave peak has propagated.

Because the soft tissue 11 is a mixture of fat and water, in actual measurement unnecessary echoes end up being detected. When this happens, there will be more noise added to the waveforms in FIG. 3, which makes it even more difficult to consistently detect the peaks.

With a conventional sonic speed measurement device, the time interval from when an ultrasonic wave is sent out until the surface propagation wave is received is measured to found the bone speed of sound. Therefore, even with a conventional sonic speed measurement device, the peaks of surface propagation waves have to be detected, but as discussed above, it is difficult to consistently detect the peaks of the surface propagation waves. For this reason, the bone sound of speed could not be consistently measured with a conventional method.

In view of this, in this embodiment a sonic speed measurement method is used that is resistant to noise, and involves integrating the waveforms of waveform signals outputted by the plurality of oscillators 24.

Specifically, when the peak positions of a plurality of waveforms coincide, if these waveforms are integrated, the amplitude of these peak positions will be larger. This property can be used to emphasize just the desired peaks so that the noise does not stand out. However, the timing at which waves reach the oscillators 24 varies considerably. To strengthen the peaks based on a particular wave, it is necessary to integrate the waveforms after shifting the phase of the waveforms so that the peaks will coincide.

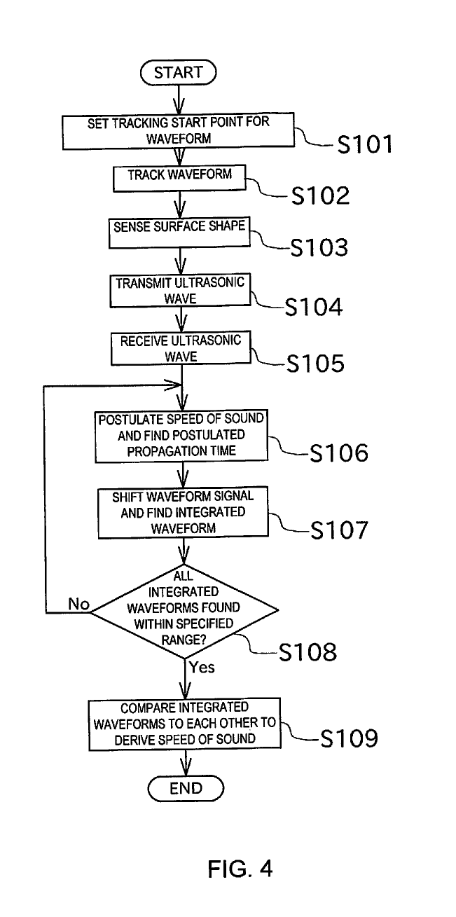

The sonic speed measurement method in this embodiment will now be described. FIG. 4 is a flowchart of the sonic speed measurement method pertaining to this embodiment. The sonic speed measurement method in this embodiment includes a waveform tracking step, a shape sensing step, a wave transmission step, a wave reception step, a postulated propagation time calculation step, a waveform integration step, and a sonic speed derivation step.

First, in the waveform tracking step, the oscillator array 22 (oscillators 24) sends out ultrasonic waves, the reflected waves thereof are received by the oscillators 24, and the resulting waveform signals are analyzed, which gives the timing at which the ultrasonic waves reflected at the surface of the cortical bone 10 was received by the oscillators 24 (S101, S102). The waveform tracking step includes a step of analyzing a waveform signal and setting a tracking start point (S101), and a step of tracking the waveform while using the set tracking start point as the origin, thereby successively determining the tracking points (S102). The tracking start point and tracking points obtained in S101 and S102 essentially refer to the timing at which the ultrasonic waves reflected at the surface of the cortical bone 10 are received by the oscillators 24. In the shape sensing step, the surface shape of the cortical bone 10 is calculated on the basis of the results of the above-mentioned waveform tracking step (S103). The waveform tracking step and the shape sensing step will be discussed in detail below.

In the wave transmission step, the dedicated wave transmission oscillator 21 sends ultrasonic waves to the cortical bone 10 (S104). In the wave reception step, the ultrasonic waves sent out from the dedicated wave transmission oscillator 21 are received by the oscillators 24 (S105), and the waveform signals shown in FIG. 3 are obtained. As shown in FIG. 3, the peaks of the surface propagation waves received by the oscillators 24 are detected later as the positions of the oscillators 24 move away from the dedicated wave transmission oscillator 21. This lag corresponds to the time (propagation time) from when the ultrasonic waves are sent out by the dedicated wave transmission oscillator 21 until the oscillators 24 receive the surface propagation waves.

Therefore, the phases of the peaks of the surface propagation waves can be brought together among the oscillators 24 by speeding up the waveform signals according to the propagation time of the surface propagation waves (by offsetting the waveform signals to the left in FIG. 3). Nevertheless, since the bone speed of sound is not known, the propagation time cannot be known ahead of time.

In this embodiment, in the postulated propagation time calculation step, the bone speed of sound is postulated, and a postulated value for the propagation time (postulated propagation time) is found for each of the oscillators 24 on the basis of this postulated bone speed of sound (sonic speed postulated value) and the surface shape of the cortical bone 10 sensed in the shape sensing step (S106).

Next, in the waveform integration step (validity index value calculation step), each waveform signal is offset by a time corresponding to the postulated propagation time, and then an integrated waveform obtained by integrating the waveform signals is found (S107). Here, the amplitude of the envelope of the integrated waveform is found as a validity index value indicating the validity of the postulated propagation time (discussed in detail below). An integrated waveform is found for each sonic speed postulated value by steadily varying the sonic speed postulated value within a specific range (a loop of S106 to S108). When the sonic speed postulated value matches the actual bone speed of sound, the phases of the peaks of the surface propagation waves of each waveform match up, and an integrated waveform is obtained in which the peaks are emphasized the most. Therefore, in the sonic speed derivation step, the point when the amplitude of the integrated waveform is largest is detected, and the sonic speed postulated value at that point is used as the measured value for the bone speed of sound (S109).

With the above method, any waves whose peak phases do not match up (that is, noise other than the peaks of the surface propagation waves) will not greatly affect the amplitude of the integrated waveform, so just the surface propagation waves can be emphasized (the surface propagation waves can be “focused”), and the bone speed of sound can be found very accurately.

Next, the configuration for implementing the above-mentioned sonic speed measurement method with the bone strength diagnostic device 1 in this embodiment will be described.

The computer 35 provided to the device main body 3 of the bone strength diagnostic device 1 shown in FIG. 1 is made up of a CPU, a RAM, a ROM, and other such hardware, and programs stored in the ROM and other such software. This hardware and software work together to allow the computer 35 to function as a waveform tracker 36, a shape sensor 40, a sonic speed sensor 50, and so forth.

First, the waveform tracker 36 and the shape sensor 40 will be described. The shape sensor 40 senses the bone surface shape that is necessary to derive the speed of sound, on the basis of the tracking result of the waveform tracker 36. That is, when the postulated propagation time is found from the postulated bone speed of sound (sonic speed postulated value), information related to the propagation path of the surface propagation waves is necessary. To find the propagation path here, the shape of the surface of the cortical bone 10 (the cross sectional contour shape of the cortical bone 10) must be acquired by one method or another. In this embodiment, the surface shape of the cortical bone 10 is sensed by the shape sensor 40 before the sonic speed sensor 50 finds the bone speed of sound.

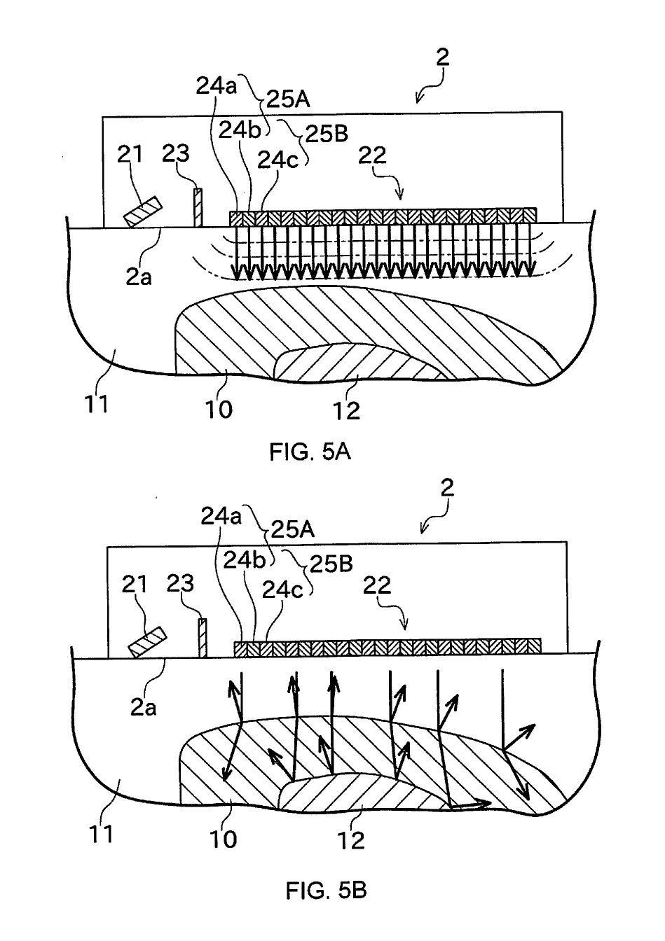

In the sensing of the bone surface shape by the shape sensor 40, the transmission of ultrasonic waves from the oscillator array 22 is performed as a preliminary stage. How the ultrasonic waves are sent out by the oscillator array 22 will be described through reference to FIG. 5. FIG. 5A shows how ultrasonic waves are sent out by the oscillator array 22, and FIG. 5B shows how the ultrasonic waves sent out by the oscillator array 22 are reflected at the front or back of cortical bone 10.

When the ultrasonic waves are sent out by the oscillator array 22, the transmission switch 32 determines the oscillator array 22 as the oscillators that will send out ultrasonic waves. A pulse signal is sent from the transmission circuit 31 to the oscillator array 22, and the plurality of oscillators 24 that make up the oscillator array 22 send out ultrasonic waves of the same phase simultaneously to the bone.

When the oscillators 24 send out waves simultaneously, this allows the planar wave shown in FIG. 5A to be generated. This planar wave is parallel to the contact face 2a, and advances through the soft tissue 11 at an orientation that is perpendicular to the contact face 2a. The planar wave is reflected as shown in FIG. 5B at the front or back of the cortical bone 10, and is received by the oscillators 24.

When the oscillators 24 receive ultrasonic waves, waveform signals corresponding to the ultrasonic waves received by the oscillators 24 are sent to the computer 35. The surface shape of the cortical bone 10 can be found by analyzing these waveform signals. These waveform signals may include not only surface reflected waves from the cortical bone 10, but also other reflected waves (such as echoes from a fascia, defects in the soft tissue, etc.). The computer 35 functions as the waveform tracker 36, and tracks the echoes of surface reflected waves from the cortical bone 10 in these waveform signals, and thereby finds the timing at which the reflected waves from the surface of the cortical bone 10 were received. In this embodiment, the waveform tracker 36 is made up of a determination index calculator 37, a tracking start point setting component 38, and a tracking execution component 39.

The determination index calculator 37 performs computation corresponding to the determination index calculation step in the waveform tracking method used by the bone strength diagnostic device 1. When a peak appears in the waveform signal received by a certain oscillator 24, the determination index calculator 37 calculates the probability that this peak is a reflected wave from the surface of the cortical bone 10 (determination index), from the characteristics appearing in this peak in the waveform signal and in the signal shape of the nearby portion. In the following description, whether the probability that a peak (echo signal) is a reflected wave from the cortical bone 10 is high or low will sometimes be expressed by saying that the peak is reliable or unreliable.

With the bone strength diagnostic device 1 in this embodiment, ultrasonic waves from the oscillators 24 are received after being reflected off an adjustment-use flat plate molded from acrylic resin, for example. The received waveform is pre-adjusted so as to be a short pulse that is sharply pointed on the positive side. The above-mentioned determination index is calculated on the basis of the idea that a wave is very likely to be a reflected wave from the surface of the cortical bone 10 if there is a large, sharply pointed peak on the positive side, or, even with a peak on the positive side that is relatively not large, if peaks on the positive side near both sides thereof appear symmetrically.

In this embodiment, there are two types of determination index that evaluate the shape of a waveform signal, and both indicate the size relation of a plurality of peaks had by an echo signal. These two types of determination index will now be described.

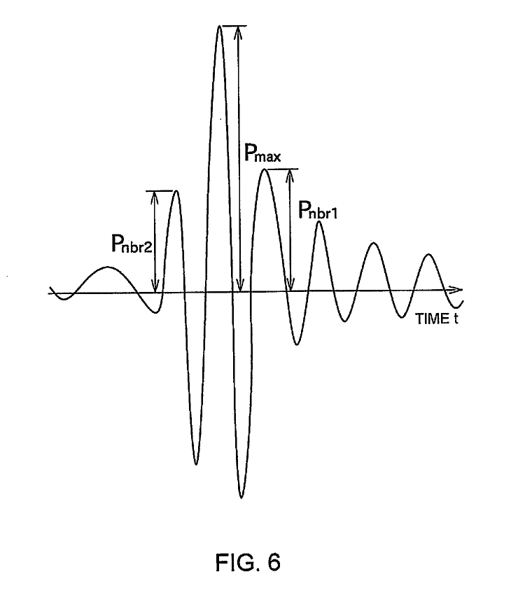

The first determination index is the ratio of the size of an adjacent peak on the positive side to the size of the maximum peak on the positive side (Pnbr1/Pmax, Pnbr2/Pmax). That is, if we let Pmax be the size of the maximum peak of the positive side, and let Pnbr1 and Pnbr2 respectively be the size of the adjacent peaks on the positive side that are adjacent on both sides in the time axis direction, if Pmax is greater than Pnbr2 and Pnbr1, as in the waveform shown in FIG. 6, then it can be concluded that the maximum peak is sharp. Based on this thinking, in this embodiment the above-mentioned ratios (Pnbr1/Pmax, Pnbr2/Pmax) were calculated for each of the peaks Pnbr1 and Pnbr2 that are adjacent on both sides, and if either is below a specific threshold (there are various possibilities, but 0.6, for example), then it is concluded that the maximum peak Pmax is very likely a reflected wave from the surface of the cortical bone 10.

The second determination index is the ratio of the absolute value of the difference between the adjacent peaks on the positive side to the size of the maximum peak on the positive side (|Pnbr1−Pnbr2|/Pmax).

This index can be considered to indicate the extent of distortion in the waveform. If the value of this ratio is large, it means that the waveform has lost its symmetry around the maximum peak, and there is a high probability that the reflecting body is recessed, or unnecessary echoes are superposed, resulting in a waveform that is inverted positive/negative and is unreliable. On the other hand, even if the ratio of the first determination index is at or above the specific threshold, if it is under a specific upper limit (such as 0.8), and the value of the second determination index is under a specific threshold (there are various possibilities, but 0.3, for example), then it is very likely that the pulse waveform has spread out somewhat in the time axis direction. Therefore, it is concluded that the maximum peak Pmax is a reflected wave from the surface of the cortical bone 10.

As discussed above, in this embodiment, the relation of the sizes of a plurality of peaks included in an echo signal is calculated as a determination index. Unlike when a correlation value is calculated as in the past, the processing load will not become excessive, so the tracking will take less time and the device can be simplified.

Here, three examples will be given of the peaks of the signal waveforms received by the oscillators 24, and how it is determined whether or not it is a reflected wave from the surface of the cortical bone 10 will be described for each.

The waveform in FIG. 6 is an example of an echo with a sharp peak that can be received by the oscillators 24. The maximum peak Pmax of this waveform is larger than the two adjacent peaks Pnbr1 and Pnbr2. When the first determination indexes (Pnbr1/Pmax and Pnbr2/Pmax) are calculated for this waveform, both are 0.6 or below. Therefore, it is concluded that there is a high probability (it is reliable) that the maximum peak Pmax of this waveform is a reflected wave from the surface of the cortical bone 10.

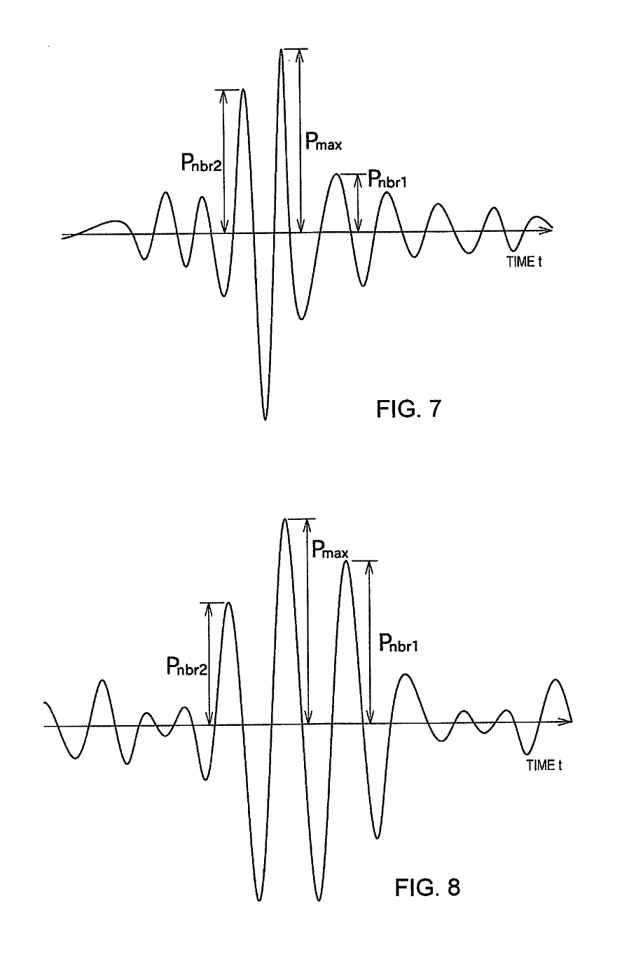

The waveform in FIG. 7 is an example of when the oscillators 24 receive an echo that has been inverted positive and negative due to one reason or another, such as that the surface shape of the cortical bone 10 that has reflected the ultrasonic waves is not flat. With this waveform, when the first determination index is calculated, the value of Pnbr1/Pmax is 0.6 or less, but the value of Pnbr2/Pmax is over 0.6 and no higher than 0.8, so the second determination index is calculated. Since the result of calculating the second determination index a Pnbr1-Pnbr2/Pmax) is over 0.3, it is concluded that there is a low probability (unreliable) that the maximum peak Pmax of this waveform is a reflected wave from the surface of the cortical bone 10.

The waveform in FIG. 8 is an example of when the oscillators 24 have received an echo waveform that has been stretched out in the time axis direction for one reason or another, although it has not undergone positive/negative inversion. With this waveform, when the first determination index is calculated, the value of Pnbr2/Pmax is 0.6 or less, but since the value of Pnbr1/Pmax is 0.8 or less and greater than 0.6, the second determination index is calculated. The result of calculating the second determination index (|Pnbr1−Pnbr2|/Pmax) is 0.3 or less, so it is concluded that there is a high probability (it is reliable) that the maximum peak Pmax of this waveform is a reflected wave from the cortical bone 10 surface.

The tracking start point setting component 38 determines the waveform tracking start point (waveform tracking start step). Here, the result of waveform tracking greatly affects the shape sensing of the shape sensor 40 (discussed below). If the tracking start point happens to be set incorrectly, there is the risk that an echo other than a reflected wave from the surface of the cortical bone 10 will be tracked, and if this happens, the tracking result ends up being virtually worthless. Therefore, to measure the speed of sound accurately, it is extremely important to set the tracking start point correctly.

The tracking start point setting component 38 in this embodiment first searches for the maximum peak on the positive side in the waveforms received by all of the oscillators 24. The tracking start point is set at the timing and for the oscillators 24 corresponding to this peak only when it is determined that there is a high probability that the peak is a reflected wave from the surface of the cortical bone 10 (very reliable).

More specifically, the determination index calculator 37 calculates the first determination index for a found peak, and if the result is 0.6 or less, it is concluded that the peak is reliable. If the first determination index is over 0.6 but no more than 0.8, and if the second determination index is no more than 0.3, it is concluded that the peak is reliable. Otherwise, the peak is concluded to be unreliable.

If the above result is that the peak is reliable, the tracking start point is set at the timing corresponding to that peak, as discussed above. If it is concluded that the peak is unreliable, the next largest peak on the positive side is then searched for among the waveforms received by all the oscillators 24. Processing to determine whether or not this peak is reliable is repeated until a reliable peak is found. Thus, the waveform tracker 36 (the tracking start point setting component 38) in this embodiment does not simply select the place where the maximum peak appears as the tracking start point, and instead selects the tracking start point only when the determination index satisfies a specific condition. As a result, serious misrecognition of shape due to tracking error can be effectively reduced.

In this embodiment, the tracking start point can be set to the signal waveform of all the oscillators 24, but what the tracking start point is set for may be limited to the signal waveform of oscillators 24 located somewhere other than at the ends in the direction in which the oscillators are lined up. The reason for this is that since the measurement object in this embodiment is the cortical bone 10, which has a rounded shape, it is believed that tracking reliability will be better if the tracking is started from a signal waveform corresponding to a middle position of the cortical bone 10.

After the tracking start point has been set as above, the tracking start point setting component 38 searches for the maximum peak on the positive side, using the waveform signal of the oscillator 24 adjacent on one side to the oscillator 24 for which was acquired the waveform signal at which the tracking start point was set (hereinafter also referred to as the start point oscillator), and the waveform signal of the oscillator 24 adjacent on the other side. When the peak is found, the tracking start point setting component 38 determines whether or not that peak is reliable. This determination is performed by using the determination index calculated by the determination index calculator 37. If the peak of an adjacent oscillator 24 is determined to be reliable, a second tracking start point is set for that peak. If the peak is determined to be unreliable, then a search is made for the peak of the waveform signal for the oscillator 24 adjacent on the opposite side from the start point oscillator, and whether or not this peak is reliable is determined. If both peaks are determined to be unreliable for the waveform signals of the oscillators 24 adjacent on both sides of the start point oscillator, then the tracking start point is to be set at another place, and the procedure starts over from the setting of the tracking start point.

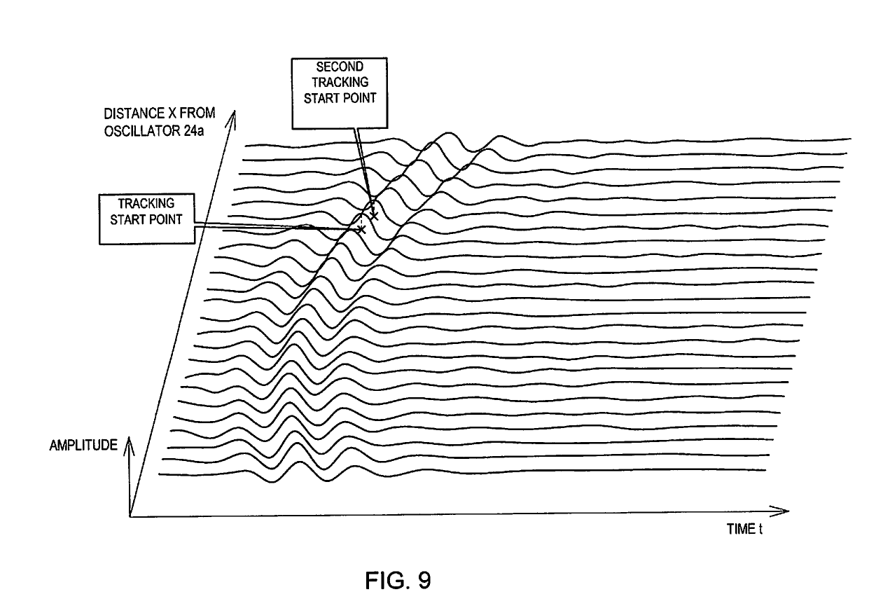

As shown in FIG. 9, the above processing allows two points to be obtained, namely, the tracking start point and the second tracking start point (in other words, a pair of tracking start points) for the signal waveform obtained by each oscillator 24. The reason for thus deciding on not one, but two tracking start points is so that a straight line for extrapolation can be drawn during tracking execution (discussed below).

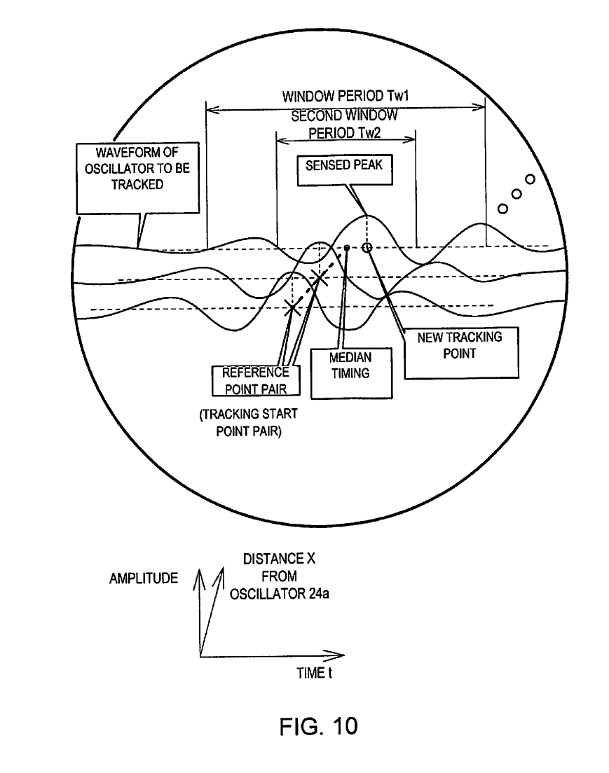

Next, the computer 35 functions as the tracking execution component 39, and waveform tracking is performed (tracking execution step). More specifically, the tracking execution component 39 uses the oscillator 24 adjacent on one side to the two oscillators 24 corresponding to the pair of tracking start points, as the oscillator 24 to be tracked. As shown in FIG. 10, when a pair of tracking start points are used as a pair of reference points, the straight line linking this pair of reference points is extended to the oscillator 24 to be tracked (straight line extrapolation), and the corresponding timing is found (hereinafter also referred to as the median timing). The tracking execution component 39 sets a specific window time Tw1 centered on the median timing, and then searches for the maximum peak on the positive side in the waveform signal of the oscillator 24 to be tracked, from among the waveforms (partial waveforms) within this window time Tw1. The length of this window time Tw1 can be variously set, but an example is a time range of about one to three times the pulse frequency.

Once the maximum peak is found, the tracking execution component 39 determines whether or not that peak is reliable. This determination is performed by using the determination index calculated by the determination index calculator 37, exactly as discussed above. If the peak is determined to be reliable, a tracking point is newly set at a timing corresponding to this peak. If the peak is determined to be unreliable, a second window time Tw2 is set that centers on the above-mentioned median timing, the maximum peak on the positive side is searched for in the waveform signal of the oscillator 24 to be tracked, from among the waveforms (partial waveforms) within this second window time Tw2, and if the size of that peak is at or above a specific threshold, a tracking point is newly set at the corresponding timing. Specifically, from the standpoint of giving priority to the continuity of tracking processing, as long as a peak is of a certain size, a tracking point is set at the location of that peak, without testing the probability that that peak is a reflected wave from the surface of the cortical bone 10. However, when tracking accuracy is taken into account, it is preferable if the above-mentioned second window time Tw2 is a shorter time interval (such as one pulse frequency or less) than the normal time window Tw1.

If it is determined that the maximum peak found within the normal window time Tw1 is unreliable, and the size of the maximum peak found within the second window time Tw2 is below a specific threshold, then the setting of the tracking point is skipped for the signal waveform of the oscillator 24 to be tracked. Even if an echo is weak, tracking errors can be made less likely to occur.

When a tracking point has been set, the oscillator 24 to be tracked is shifted one point away from the pair of tracking start points, and the same processing as above is performed by using the tracking point that was just newly set, and the tracking start point on one side as a reference point pair. The straight line that links this reference point pair is extended to the oscillator 24 to be tracked, and a specific window time Tw1 is set centered on the median timing thus obtained. A search is made for the maximum peak on the positive side for the waveform signal of the oscillator 24 to be tracked, within the range of this window time Tw1. Whether or not this peak is reliable is checked, and if it is reliable, a tracking point is set at the timing corresponding to that peak. If it is unreliable, a second window time Tw2 is set centered on the above-mentioned median timing, and a search is made for the maximum peak on the positive side for the waveform signal of the oscillator 24 to be tracked, within this second window time Tw2. If the size of the peak is at or above a specific threshold, a tracking point is set at the corresponding timing.

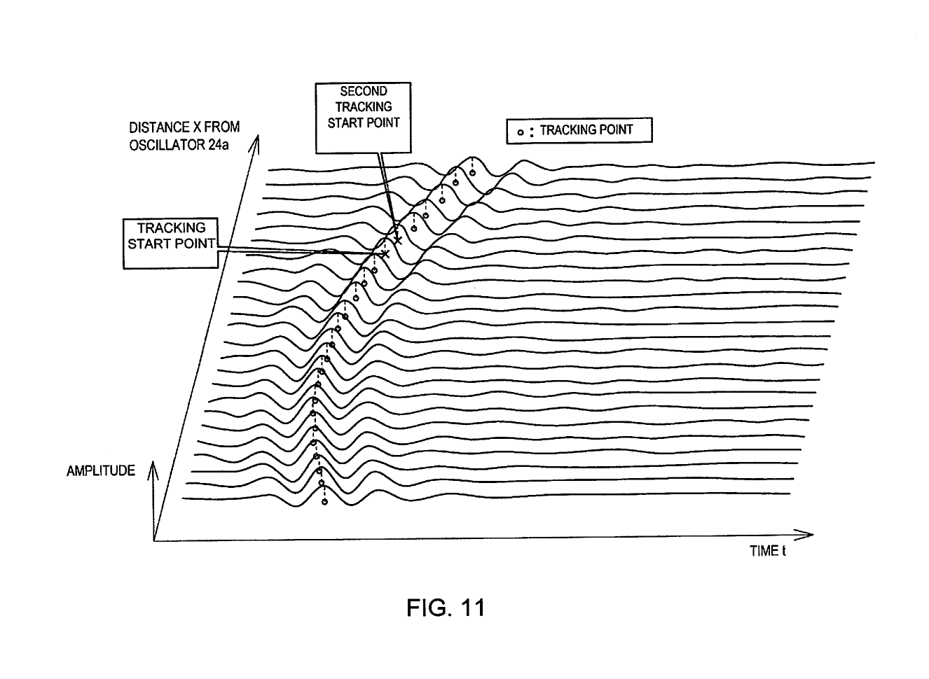

In the above processing, the reference point pair and the oscillator to be tracked are shifted by one each, while the propagate at which the tracking point can be set is found. This is repeated until failure to find this peak has continued for a specific number of times in a row. This search allows waveform tracking to be performed as shown in FIG. 11. This waveform tracking is performed not only for the oscillators 24 that are lined up on one side as viewed from the pair of tracking start points, but also for the oscillators 24 that are lined up on the other side.

As discussed above, the waveform tracker 36 (the tracking execution component 39) in this embodiment is configured so that tracking points are set while taking into account both a determination index and tracking continuity. Therefore, robust tracking can be accomplished even when a signal waveform includes an unnecessary echo, and the timing at which ultrasonic waves were reflected by the surface of the cortical bone 10 can be consistently found. In particular, in this embodiment, as a general rule tracking points are set while taking a determination index into account, so even if the above-mentioned window time Tw1 is increased, the tracking will be unlikely to go past the targeted echo to an unnecessary echo. It is possible to achieve both flexibility that allows faithful tracking without losing continuity even with waveforms based on complicated bone shapes, and accuracy that will not result in tracking errors even with unnecessary echoes.

Next, the computer 35 functions as the shape sensor 40, to sense the angle and time when reflected waves were received by the oscillators 24, using the waveform tracking result discussed above as a basis, and to find the surface shape of the cortical bone 10 on the basis of this.

More specifically, the shape sensor 40 is made up of an arrival direction sensor 41, a surface reflection point sensor 43, and a bone surface line sensor 44.

First, the arrival direction sensor 41 will be described. The arrival direction sensor 41 selects oscillator groups 25 as groups of two adjacent oscillators out of the plurality of oscillators 24, and senses the direction in which the ultrasonic waves arrive at the oscillator groups 25. In the following description of the shape sensor 40, ultrasonic waves received after plane waves sent from the oscillator array 22 have been reflected at the front of the cortical bone 10 will sometimes be called surface reflected waves, and ultrasonic waves received after the same have been reflected at the back of the cortical bone 10 will sometimes be called rear reflected waves. If the oscillator groups 25 need to be distinguished from one another, they may be referred to as the oscillator group 25A, oscillator group 25B, etc., adding an upper-case letter as a suffix, starting from the side closest to the dedicated wave transmission oscillator 21.

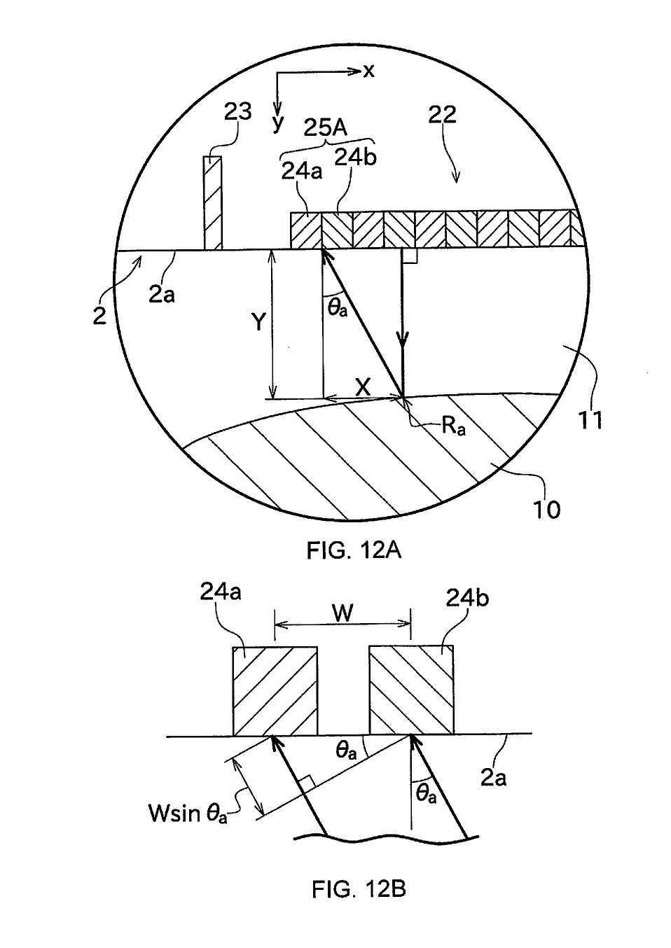

This will now be described in specific terms through reference to FIG. 12. FIG. 12A is a simplified detail view of the area near the oscillator group 25A receiving surface reflected waves, and FIG. 12B is a simplified diagram illustrating the difference in the propagation paths of surface reflected waves reaching two oscillators 24a and 24b that make up an oscillator group. With a given oscillator group 25, the directions in which surface reflected waves arrive at two adjacent oscillators 24 are similar. For example, in FIG. 12, the surface reflected waves can be considered to have arrived at an arrival angle θa at the oscillator 24a and the oscillator 24b that make up the oscillator group 25A. The following computation is performed to find the arrival angle θa.

First, the arrival direction sensor 41 measures a time difference Δt at which the two oscillators 24a and 24b that make up the oscillator group 25A sense the peak of a surface reflected wave. As discussed above, surface reflected waves and rear reflected waves are generated when plane waves are sent out from the oscillator array 22. Since a surface reflected wave is always received before a rear reflected wave, the peak of a surface reflected wave can be properly detected.

Then, the arrival angle θa of the surface reflected wave for the oscillator group 25A is found on the basis of this time difference Δt. As shown in FIG. 12B, if we let W be the spacing between the oscillator 24a and the oscillator 24b, a surface reflected wave reaches the oscillator 24a after propagating a distance that is W sin θa longer than that of the oscillator 24b. If we let SOS_{soft} be the speed of sound through soft tissue, we obtain the following.

SOS_{soft}Δt=W sin θa

Therefore, the arrival angle θa can be found as follows.

θa=arcsin(SOS_{soft}Δt/W)

The arrival direction sensor 41 similarly finds the arrival angle for the other oscillator groups 25. In this embodiment, a value obtained experimentally is used as the speed of sound SOS_{soft} through the soft tissue 11, but a measured value may be used instead.

An arrival time sensor 42 will now be described. The arrival time sensor 42 finds the arrival time Ta, which is how long it takes for a surface reflected wave to reach the oscillator group 25 after an ultrasonic wave is sent out by the oscillator array 22. In this embodiment, the arrival time Ta is an average value for the time it takes for surface reflected waves to reach the two oscillators 24 in an oscillator group 25 after an ultrasonic wave is sent out by the oscillator array 22. An average value need not be used, however, and the arrival time Ta may instead be how long it takes for a surface reflected wave to reach either one of the oscillators 24.

The surface reflection point sensor 43 will now be described. The surface reflection point sensor 43 senses a reflection point Ra of a surface reflected wave that has reached an oscillator group 25, on the basis of the arrival angle θa and the arrival time Ta.

In the plane shown in FIG. 12, the x axis is the direction in which the oscillator array 22 is arranged, and the y axis is a direction perpendicular to the x axis. We will let X be the distance from the oscillator group 25A to the reflection point Ra in the x axis direction, and let Y be the distance in the y axis direction. As is clear from FIG. 12, the propagation distance La of the surface reflected wave is as follows.

La=Y+Y/cos θa

Meanwhile, if we use the arrival time Ta and the speed of sound SOS_{soft} through the soft tissue 11, we obtain the following.

La=SOS_{soft}×Ta

Therefore, the distances X and Y indicating the position of the reflection point Ra can be found as follows.

Y=SOS_{soft}×Ta×cos θ/(1+cos θ)

X=Y×tan θ=SOS_{soft}×Ta×sin θ/(1+cos θ)

The location of the reflection point Ra can be calculated on the basis of the arrival time Ta and the arrival angle θa of a plane wave. The surface reflection point sensor 43 similarly finds the reflection points for the other oscillator groups 25.

The bone surface line sensor 44 senses a bone surface line by connecting the plurality of reflection point found by the surface reflection point sensor 43 with a curved or straight line. Since the reflection points are points on the surface of the cortical bone 10, the bone surface line represents the surface shape of the cortical bone 10. The surface shape (bone surface line) of the cortical bone 10 can be obtained by the shape sensor 40 as above.

Next, the sonic speed sensor 50 will be described. The sonic speed sensor 50 senses the speed of sound through the cortical bone 10 (bone speed of sound). Prior to the sensing of the bone speed of sound by the sonic speed sensor 50, waveform tracking is performed by the waveform tracker 36 (waveform tracking step), and the sensing of the bone surface line is performed by the shape sensor 40 (shape sensing step). The transmission of ultrasonic waves is then performed by the dedicated wave transmission oscillator 21 (transmission step), and the returning ultrasonic waves are received by the oscillator array 22, and waveform signals are sent to the computer 35 (reception step). The bone speed of sound is derived by the sonic speed sensor 50 on the basis of the waveform signals of the oscillators 24.

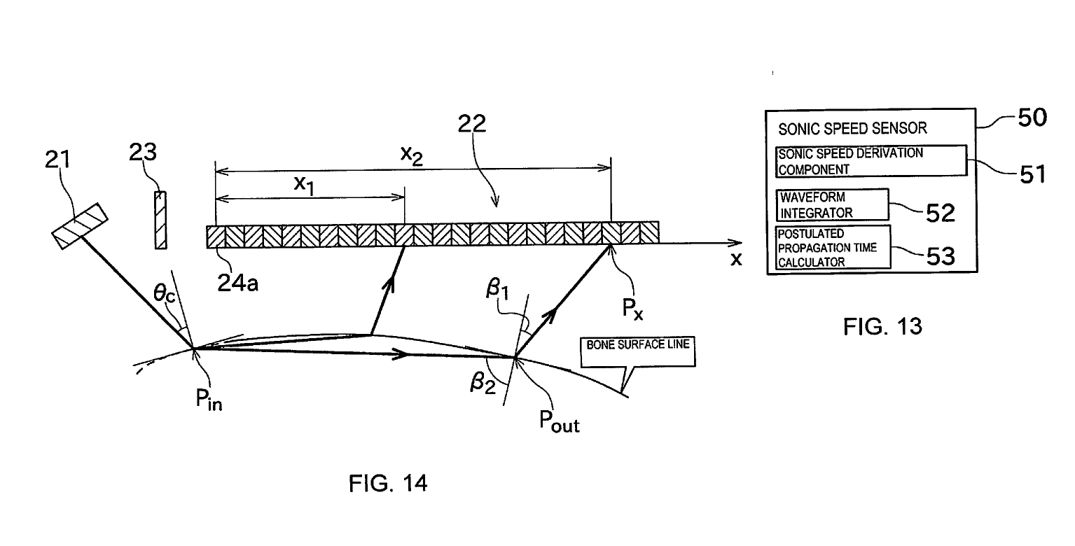

More specifically, as shown in FIG. 13, the sonic speed sensor 50 is made up of a postulated propagation time calculator 51, a waveform integrator 52, and a sonic speed derivation component 53. The postulated propagation time calculator 51 performs computation processing corresponding to the above-mentioned postulated propagation time calculation step. Specifically, the postulated propagation time calculator 51 postulates the bone speed of sound, and finds the postulated propagation time on the basis of this postulated bone speed of sound. The postulated propagation time calculator 51 first finds the propagation path of a surface propagation wave in order to find the postulated propagation time. If the surface shape of the cortical bone 10, the bone speed of sound, and the speed of sound through the soft tissue 11 are already known, then the path over which the surface propagation waves propagate from the dedicated wave transmission oscillator 21 to the oscillators 24 can be uniquely found by the known Snell's law.

Here, the bone shape is represented by the bone surface line sensed by the shape sensor 40. The bone speed of sound is used in calculation by postulating a suitable value (postulated bone speed of sound) from within a specific range set on the basis of experimental values for the bone speed of sound. The speed of sound through the soft tissue 11 is an experimental value in this embodiment, but a pre-measured value may be used instead. The speed of sound through the soft tissue 11 can also be measured by sending ultrasonic waves from the dedicated wave transmission oscillator 21 or the oscillator array 22, and receiving them with the oscillator array 22.

As described above, there are two kinds of surface propagation wave received by the oscillators 24, depending on the propagation path: a leaky surface wave and a surface refracted wave. In this embodiment, the description will be of calculating the propagation path of a surface refracted wave.

The description will now refer to FIG. 14. FIG. 14 is a simplified diagram illustrating a method for calculating the propagation path of a surface reflected wave. The postulated propagation time calculator 51 first determines an incident point Pᵢₙ at which an ultrasonic wave from the dedicated wave transmission oscillator 21 is incident in the interior of the cortical bone 10, in order to determine the propagation path of a surface refracted wave. Since the incident point Pᵢₙ is located somewhere other than directly under the oscillator array 22, with a method in which plane waves are sent out from the oscillator array 22, and surface reflected waves are sensed (sensing of the cortical bone surface shape by the shape sensor 40), the surface shape near the incident point Pᵢₙ cannot be acquired. In this embodiment, the shape near the incident point Pᵢₙ is predicted on the basis of the bone surface line found by the shape sensor 40. In FIG. 14, the predicted bone surface line is shown as a dotted line.

Next, the postulated propagation time calculator 51 finds the location where the ultrasonic waves are incident on this predicted line (incident point Pᵢₙ). The ultrasonic waves can be incident in the interior of the cortical bone 10 at any angle smaller than a critical angle θc. However, surface refracted waves will be received by the oscillators 24 only when the ultrasonic waves are incident in the cortical bone 10 at an incidence angle that is close to the critical angle θc. In this embodiment, the incidence location is found by approximating that the ultrasonic waves are incident in the cortical bone 10 at the critical angle θc. The critical angle θc is determined by the bone speed of sound and the speed of sound through the soft tissue, so the incident point Pᵢₙ can be uniquely found by postulating the bone speed of sound.

Then, the postulated propagation time calculator 51 sets an arbitrary point Pₒᵤₜ on the bone surface line. The linear distance from the incident point Pᵢₙ to the point Pₒᵤₜ is the distance over which the ultrasonic waves propagate through the cortical bone 10. The postulated propagation time calculator 51 finds the angle when the ultrasonic waves are emitted on the soft tissue 11 from Pₒᵤₜ. The angle when the ultrasonic waves are emitted can be uniquely found by Snell's law. More specifically, if we let SOS_{bone} be the postulated speed of sound, and SOS_{soft} be the speed of sound through soft tissue, then the relation between a refraction angle β1 and an incidence angle β2 when ultrasonic waves are emitted from the cortical bone 10 to the soft tissue 11 side is as follows.

sin β1/SOS_{soft}=sin β2/SOS_{bone}

The incidence angle β2 can be found from the slope of the straight line PᵢₙPₒᵤₜ and the shape of the bone surface line. Therefore, the refraction angle β1 can be found from the above equation.

As shown in FIG. 14, the x axis lies in the direction in which the oscillator array 22 is arranged, using as the origin the location of the oscillator 24a closest to the dedicated wave transmission oscillator 21. Since we know the refraction angle β1 and the shape of the bone surface line, ultrasonic waves emitted from the point Pₒᵤₜ intersects the x axis at a point Px. The propagation path of the surface refracted waves from the dedicated wave transmission oscillator 21 to the point Px can be found by the computation processing described above.

In finding the incident point Pᵢₙ, the purpose of approximating that the ultrasonic waves are incident in the cortical bone 10 at the critical angle θc as mentioned above is to facilitate calculation, and the calculated propagation path is an approximate value. On the other hand, it is also possible to calculate the propagation path exactly, without approximating the incidence angle on the cortical bone 10 at the critical angle θc. If the surface shape of the cortical bone 10 is already known, Snell's law can be used twice while varying the incidence angle on the cortical bone 10, allowing the propagation path from the dedicated wave transmission oscillator 21 to the point Px to be calculated exactly. In this case, the incident point Pᵢₙ varies with the point Px. However, this exact calculation of the propagation path takes longer for processing than a calculation method in which the incidence angle is approximated with the critical angle θc. In this embodiment, the configuration is such that the propagation path can be calculated by selecting either the approximated calculation method or the exact calculation method.

The postulated propagation time calculator 51 finds the propagation time from when the ultrasonic waves are sent out by the dedicated wave transmission oscillator 21 until the surface refracted waves reach the point Px, on the basis of the propagation path thus found. This is expressed as follows.

(propagation time until point Px)=(distance propagated through soft tissue)×SOS_{soft}+(distance propagated through cortical bone)×SOS_{bone}

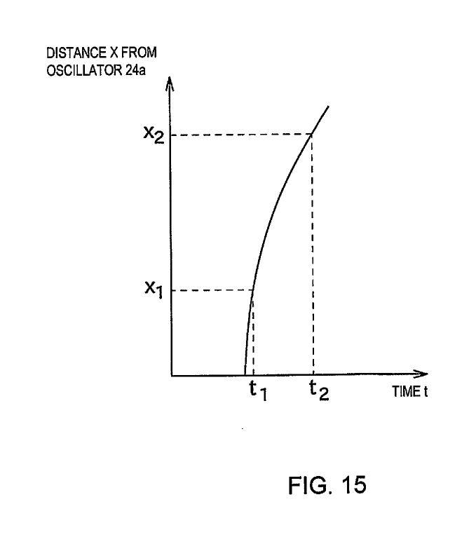

The postulated propagation time calculator 51 repeats the above-mentioned propagation time calculation while the position of Pₒᵤₜ is varied. Since the propagation time of the surface refracted waves up to the point Px in each case is found while the position of the point Px is varied, the t-x curve shown in FIG. 15 can be found. The vertical axis x in FIG. 15 is the distance from the oscillator 24a, and the horizontal axis t is the propagation time from when the dedicated wave transmission oscillator 21 sends out an ultrasonic wave until a surface refracted wave reaches the x axis.

Finally, the postulated propagation time calculator 51 finds the time it takes for a surface refracted wave to reach each of the oscillators 24 (postulated propagation time). Since the x coordinate of the oscillators 24 (the distance from the oscillator 24a) is known, the postulated propagation time of the oscillators 24 can be found by referring to the t-x curve.

Next, the waveform integrator 52 will be described. The waveform integrator (validity index value calculator) 52 performs computation processing corresponding to the above-mentioned waveform integration step. The waveform integrator 52 offsets the waveforms of the waveform signals outputted by the oscillators 24 by the postulated propagation time, and integrates them to find an integrated waveform.

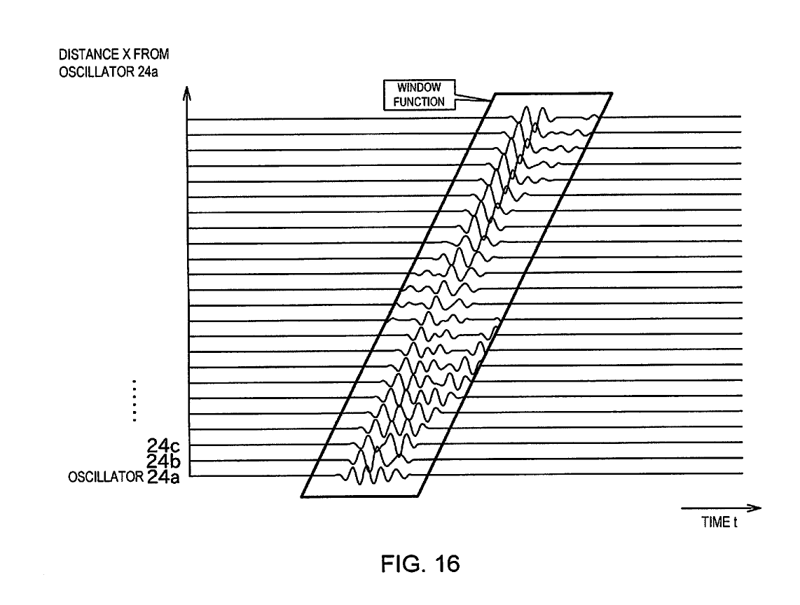

A specific description will now be given. First, the waveform integrator 52 multiplies each of the waveform signals by a suitable window function to cancel out waves other than surface refracted waves prior to integrating the waveform signals (FIG. 16). To decide on the range of the window function, the bone speed of sound and the propagation time have to be known ahead of time to a certain extent. However, even if all of the waves other than surface refracted waves cannot be cancelled out by the window function, the influence of remaining waves can be weakened by integration of the waveform signals. Therefore, there is no need to determine the window function exactly so as to completely cancel out waves other than surface refracted waves. The window function need only be determined by allowing a suitable margin, on the basis of experimental values for the bone speed of sound and the speed of sound through the soft tissue 11, etc.

The amplitude of the waveform signals weakens as the oscillators 24 move away from the dedicated wave transmission oscillator 21. The waveform integrator 52 multiplies the waveform signals of the oscillators 24 by a suitable gain to adjust the amplitudes of the waveform signals to be about the same. The method for multiplying the gain here may involve determining on the basis of the greatest amplitude of the waveform signals obtained by the window function, or may be involve determining by postulating the attenuation of a suitable exponential function.

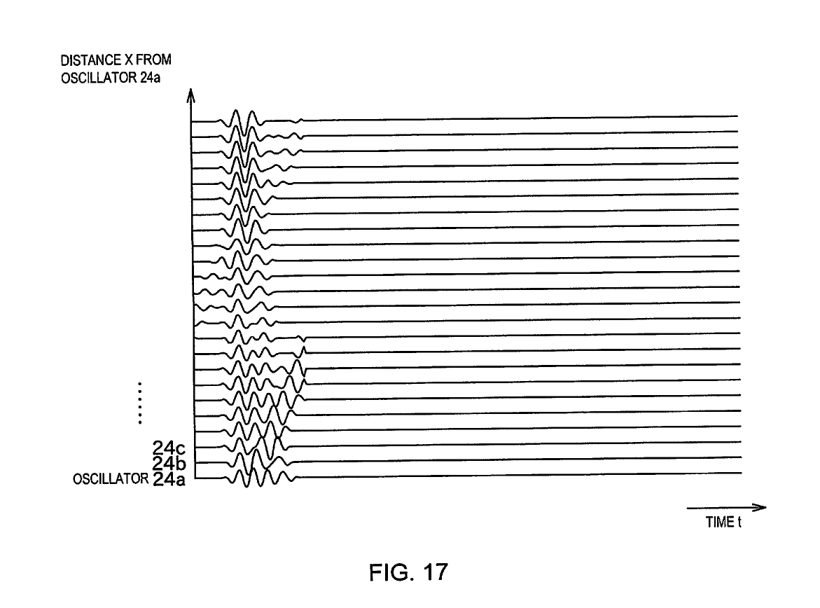

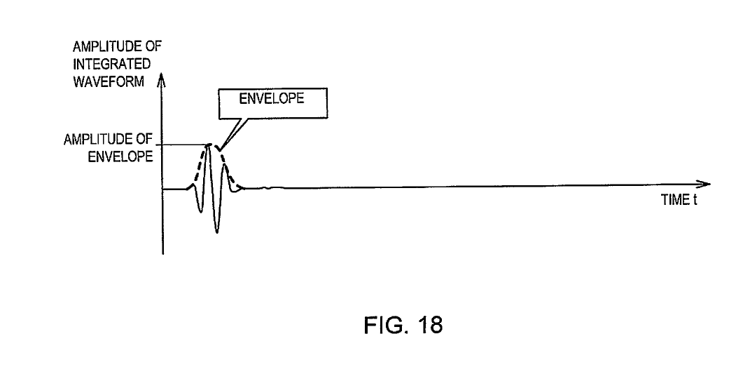

Then, the waveform integrator 52 shifts the waveform signals adjusted by multiplication with the window function and gain so as to advance the time by an amount corresponding to the postulated propagation time of the oscillators 24 (that is, it shifts them to the left in FIG. 16). When the waveform signals are shifted, the result is as shown in FIG. 17, for example. In FIG. 17, the phases of the peaks of the surface refracted waves included in the waveform signals match up. This matching up of the peaks as a result of shifting the waveform signals is a situation in which the postulated speed of sound matches the actual bone speed of sound. The waveform integrator 52 finds the integrated waveform shown in FIG. 18 by integrating the shifted waveform signals.

Finally, the waveform integrator 52 produces an envelope of the integrated waveform, and finds the amplitude of this envelope (see FIG. 18). As discussed below, the amplitude of this envelope is an index of whether or not the peaks of the surface propagation waves match up with each other after offsetting (to put this another way, whether or not the postulated propagation time matches the actual bone speed of sound). The amplitude of the envelope is a validity index value indicating the validity of the postulated propagation time.

The sonic speed derivation component 53 will now be described. The sonic speed derivation component 53 calls up the functions of the postulated propagation time calculator 51 and the waveform integrator 52, and is configured to repeat processing (loop processing) for finding the amplitude of the envelope.

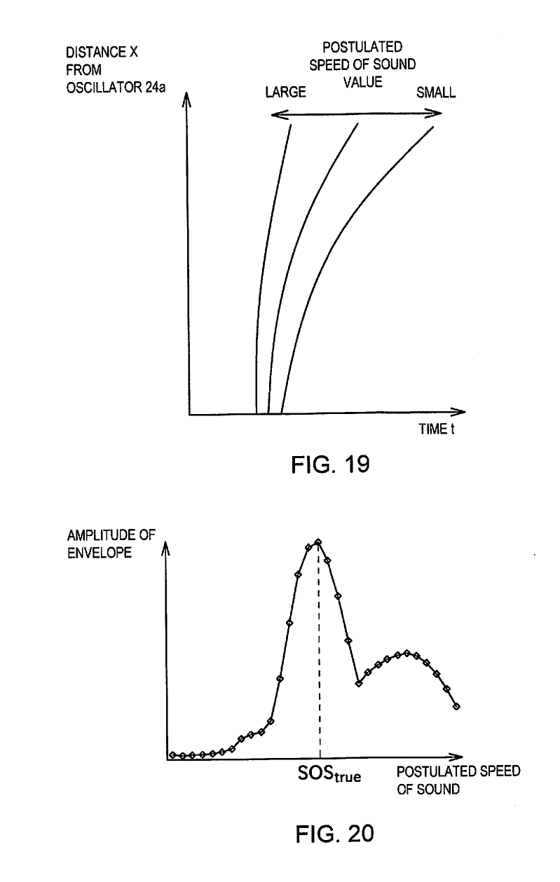

In the above-mentioned loop processing performed by the sonic speed derivation component 53, the postulated propagation time calculator 51 calculates the postulated propagation time by using a postulated speed of sound value that is different from the previous time, every time there is a call-up. The postulated propagation time calculator 51 finds a new t-X curve by using a new postulated speed of sound value for every loop. For example, FIG. 19 shows a plurality of t-x curves found by varying the postulated speed of sound value. As shown in the graph of FIG. 19, when the postulated speed of sound value is different, the slope of the t-x curve, the intersection between this t-x curve and the t coordinate axis, and so forth change. If the postulated speed of sound value is different, then the amount by which the waveform signals are shifted in integrating the waveform signals is also different.

When the amount by which the waveform signals are shifted changes, the integrated waveform also varies. Therefore, the amplitude of the envelope changes when the postulated speed of sound is changed. The sonic speed derivation component 53 repeats the above-mentioned loop while changing the postulated speed of sound within a specific range, and thereby finds the envelope amplitude values for every situation within this specific range. FIG. 20 shows an example of the result of finding and plotting the envelope amplitude while thus varying the postulated speed of sound.

In the example in FIG. 20, the amplitude of the envelope is greatest when the postulated speed of sound is SOSₜᵣᵤₑ. At SOSₜᵣᵤₑ, it is believed that the peaks of the surface refracted waves included in the waveform signals are also strengthened the most (as in FIG. 17, the phases of the peaks of the surface refracted waves match up). The sonic speed derivation component 53 employs this SOSₜᵣᵤₑ as the measured value for the speed of sound. As discussed above, the sonic speed derivation component 53 derives the speed of sound by performing computation processing corresponding to the sonic speed derivation step.

As described above, the bone strength diagnostic device 1 in this embodiment comprises the oscillators 24, the determination index calculator 37, the tracking start point setting component 38, and the tracking execution component 39. The oscillators 24 are lined up in order to acquire echo signals for the ultrasonic beams sent out. The determination index calculator 37 calculates a determination index that represents the probability that these echo signals are reflected waves from the surface of the cortical bone 10. The tracking start point setting component 38 sets a pair of tracking start points at the timing of the signal waveforms corresponding to an echo signal for which the determination index satisfies a specific condition, received by two oscillators 24 of the plurality of oscillators 24. The tracking execution component 39 successively sets tracking points for the echo signals acquired from other oscillators 24, using the tracking start points as the origin. If the determination index does not satisfy a specific condition, no tracking start point is set, which prevents the tracking start points from being set incorrectly. As a result, this prevents mistakes in which an unintended echo is tracked, affording more robust waveform tracking.

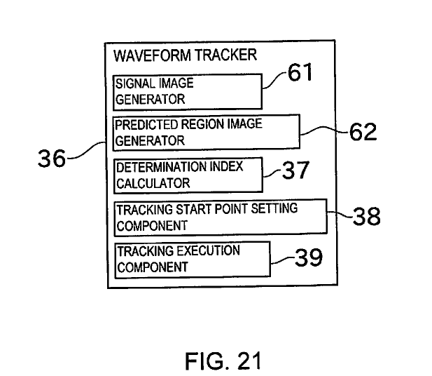

A second embodiment will now be described. FIG. 21 is a function block diagram showing the configuration of the waveform tracker 36 in the bone strength diagnostic device in a second embodiment. In the description of this embodiment, those members that are the same as or similar to those in the above-described embodiment will be numbered the same and will not be described again here.

As shown in FIG. 21, the bone strength diagnostic device in the second embodiment comprises a signal image generator 61 and a predicted region image generator 62 in the waveform tracker 36, in addition to comprising the determination index calculator 37, the tracking start point setting component 38, and the tracking execution component 39, just as in the first embodiment.

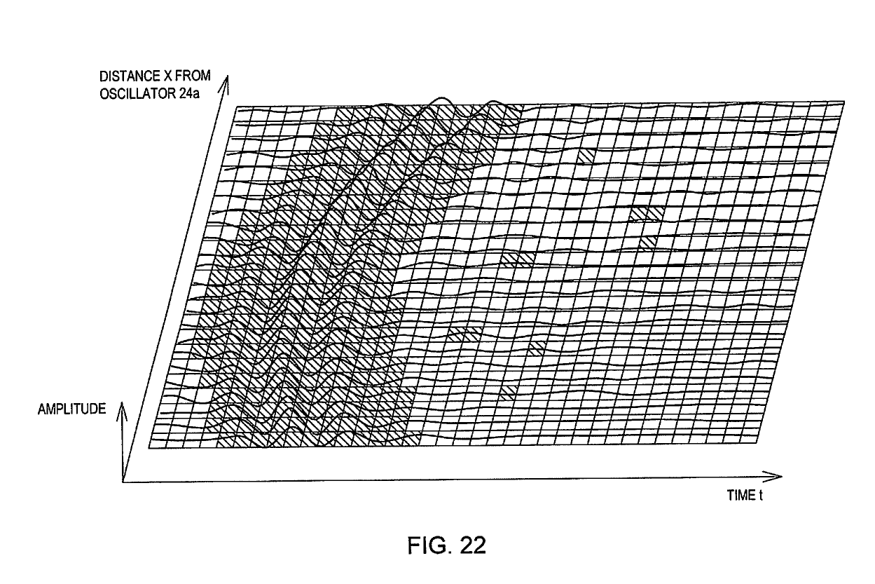

The signal image generator 61 divides the signal waveforms acquired by the plurality of oscillators 24 into a plurality of parts in the time axis direction, digitizes (binarizes) these, and arranges them in a matrix to produce a signal image. More specifically, in each of the divided time segments, it is determined whether or not the envelope of the signal waveform has gone over a specific threshold, a “1” being assigned if it has gone over, and a “0” if it has not, which produces a binary image. FIG. 22 shows an example in which a binary image is produced form the signal waveforms acquired by the oscillators 24. In FIG. 22, squares with hatching mean a pixel of “1,” while squares with no hatching mean a pixel of “0.”

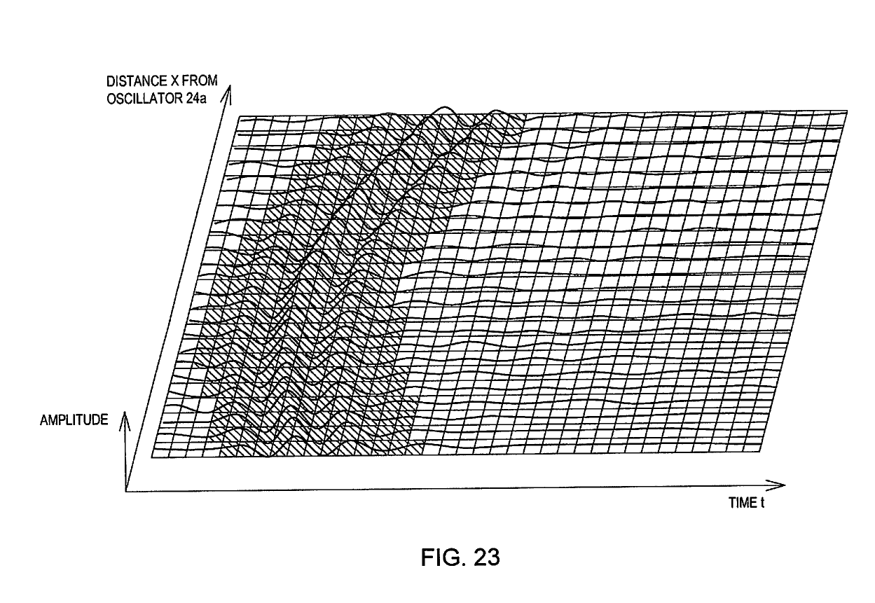

The predicted region image generator 62 obtains a predicted region image indicating a region where it is highly probable that an echo signal corresponding to the surface shape of the cortical bone 10 is included, by performing suitable image processing on the signal image (binary image) obtained by the signal image generator 61. Various kinds of image processing can be performed by the predicted region image generator 62, but in this embodiment, processing is performed in which small, independent pixels of “1” are removed. FIG. 23 shows an example of a predicted region image obtained as a result of the image processing by the predicted region image generator 62.

With the waveform tracker 36 in this embodiment, the setting of tracking start points or tracking points is performed only in the time segments and for the oscillators 24 (waveform signals) corresponding to pixels of “1” in the above-mentioned predicted region image, at the tracking start point setting component 38 and the tracking execution component 39. More specifically, when a window period has been set in a portion where pixels of “1” are continuous, the setting of tracking start points or tracking points is limited to within this window period. Processing that skips the setting of tracking points is possible in portions corresponding to pixels of “0” (where there is a low probability that an echo signal of the surface of the cortical bone 10 is included). This more effectively prevents mistakes in which echoes other than from the surface of the cortical bone 10 end up being tracked.

A method in which an image is used to raise tracking accuracy as in this embodiment is more advantageous than the configuration in the first embodiment above in that signal waveforms received by the oscillators 24 can be taken into account comprehensively in the form of an image. The above-mentioned signal image and predicted region image are only utilized in the interior of the device in this embodiment, but can also be outputted to a suitable output device (a display, a printer, etc.).

Preferred embodiments of the present invention were described above, but the above configurations can be modified as follows, for example.

The calculation of a determination index is not limited to the above-mentioned two types, and may be calculated by some other method. For instance, the determination index may be calculated by taking into account the size of the peaks located adjacent to the adjacent peaks. However, the determination index is preferably calculated so that the probability that an echo signal is a reflected wave from the cortical bone 10 will be higher if the peak that appears in the waveform of an echo signal is strong, or if the peak is sharp, or if there is little distortion in the waveform. In the above embodiments, waveform tracking was performed using the peak on the positive side of the waveform, but waveform tracking may instead be performed using the peak on the negative side of the waveform. In the above embodiments, the tracking start points and tracking points were set at a timing corresponding to the maximum peak of the waveform in an echo signal, but the tracking start points and tracking points may instead be set at some other characteristic point of the echo signal, such as the zero cross point after the maximum peak. The tracking start points may also be found individually, instead of as a pair as in the above embodiments.

The computer 35 may be changed so that it is provided on the ultrasonic wave transceiver 2 side. Also, the ultrasonic wave transceiver 2 and the device main body 3 are not limited to a configuration in which they are provided separately, and the ultrasonic wave transceiver 2 and the device main body 3 may instead be integrated. The waveform tracker 36 may track rear reflected waves in addition to tracking surface reflected waves from the cortical bone 10. The shape sensor 40 may sense the rear face shape in addition to the surface shape of the cortical bone 10. In this case, the cortical bone thickness can be found on the basis of the surface shape and the rear face shape. The cortical bone thickness thus found can be used as an index of the soundness of the bone.

The waveform tracking device of the present invention is not limited to an application in which the speed of sound through bone is measured, and can be widely applied to non-destructive testing and so forth in which ultrasonic waves are used, for example.

1. A waveform tracking device comprising:
a plurality of wave receivers disposed in a line to acquire echo signals of a transmitted ultrasound beam;
a determination index calculator configured to calculate a determination index expressing a probability that the echo signal is a reflected wave from a measurement object;
a tracking start point setting component configured to set a tracking start point at a signal waveform timing corresponding to the echo signal for which the determination index satisfies a specific condition and that is received by a part of the plurality of wave receivers; and
a tracking execution component configured to successively set tracking points for echo signals acquired from other wave receivers using the tracking start point as an origin.

2. The waveform tracking device according to claim 1, wherein
when the determination index satisfies a specific condition for an echo signal with a maximum peak out of the echo signals acquired by the plurality of wave receivers, the tracking start point setting component sets the tracking start point at a signal waveform timing corresponding to the echo signal.

3. The waveform tracking device according to claim 1, wherein
the determination index is calculated so that the probability that the echo signal is a reflected wave from a measurement object becomes higher in at least one of the following cases: a peak expressed by a waveform of the echo signal is strong, the peak is sharp, and there is little distortion in the waveform.

4. The waveform tracking device according to claim 2, wherein
the determination index is calculated so that the probability that the echo signal is a reflected wave from a measurement object becomes higher in at least one of the following cases: a peak expressed by a waveform of the echo signal is strong, the peak is sharp, and there is little distortion in the waveform.

5. The waveform tracking device according to claim 1, wherein
at least a value that evaluates a size relation between a plurality of peaks had by the echo signals is used as the determination index.

6. The waveform tracking device according to claim 2, wherein
at least a value that evaluates a size relation between a plurality of peaks had by the echo signals is used as the determination index.

7. The waveform tracking device according to claim 6, wherein
at least a value that evaluates a relation between a size of a maximum peak that is a largest peak out of a plurality of peaks had by the echo signals, and a size of an adjacent peak adjacent to this maximum peak is used as the determination index.

8. The waveform tracking device according to claim 6, wherein
when a largest peak out of a plurality of peaks had by the echo signals is a maximum peak, a peak adjacent to the maximum peak on one side is one side adjacent peak, and a peak adjacent to the maximum peak on the other side is the other side peak, at least a value that evaluates a relation between a size of the maximum peak and a difference between a size of the one side adjacent peak and a size of the other side adjacent peak is used as the determination index.

9. The waveform tracking device according to claim 1, wherein
the tracking start point setting component sets the tracking start point at the signal waveform timing corresponding to the echo signal acquired by a wave receiver that is not at an end of the line of the wave receivers.

10. The waveform tracking device according to claim 2, wherein
the tracking start point setting component sets the tracking start point at the signal waveform timing corresponding to the echo signal acquired by a wave receiver that is not at an end of the line of the wave receivers.

11. The waveform tracking device according to claim 1, wherein
the tracking execution component
takes out a partial waveform by setting a specific window period in a time axis direction centered on a point corresponding to the tracking start point or the tracking point from an adjacent signal waveform that is a signal waveform received by a wave receiver adjacent to the wave receiver that acquires an echo signal to which the tracking start point or the tracking point belongs,
checks whether or not an echo signal that satisfies the determination index exists in this partial waveform, and
sets the tracking point at a signal waveform timing corresponding to this echo signal if exists.

12. The waveform tracking device according to claim 11, wherein
the tracking execution component
takes out a partial waveform by setting a specific second window period in a time axis direction centered on a point corresponding to the tracking start point or the tracking point from the adjacent signal waveform if no echo signal that satisfies the determination index exists in the partial waveform, and
sets the tracking point at a signal waveform timing corresponding to the maximum peak in this partial waveform, and
the second window period is set to be a shorter time than the window period.

13. The waveform tracking device according to claim 12, wherein
the tracking execution component skips setting of a tracking point with respect to the adjacent signal waveform if the maximum peak in the partial waveform taken out by setting the second window period is smaller than a specific size.

14. The waveform tracking device according to claim 1, further comprising
a signal image generator configured to divide signal waveforms acquired by the plurality of wave receivers into a plurality of parts in the time axis direction, respectively, to digitize them, and configured to arrange them in a matrix to obtain a signal image, and
a predicted region image generator configured to subject the signal image to image processing to obtain a predicted region image indicating a region where there is a high probability that an echo signal corresponding to a shape of the measurement object is included,
the tracking start point setting component setting the tracking start point only for an echo signal of a time period and the wave receiver corresponding to the region indicated by the predicted region image.

15. The waveform tracking device according to claim 14, wherein
the tracking execution component sets the tracking point only for an echo signal of the time period and the wave receiver corresponding to the region indicated by the predicted region image.

16. The waveform tracking device according to claim 1, further comprising
a shape sensor configured to sense a shape of the measurement object on the basis of the tracking result.

17. The waveform tracking device according to claim 16, further comprising
a sonic speed sensor configured to find a speed of sound through the measurement object by using the shape sensed by the shape sensor.

18. An ultrasound diagnostic device comprising:
a plurality of wave receivers configured to send out an ultrasound beam toward an interior of a subject, and configured to receive echo signals of the ultrasound beam;
a determination index calculator configured to calculate a determination index expressing a probability that the echo signals is a reflected wave from a measurement object;
a tracking start point setting component configured to set a tracking start point at a signal waveform timing corresponding to the echo signal for which the determination index satisfies a specific condition and that is received by a part of the plurality of wave receivers; and
a tracking execution component configured to successively set tracking points for the echo signals acquired from other wave receivers using the tracking start point as an origin.

19. The ultrasound diagnostic device according to claim 18, wherein
when the determination index satisfies a specific condition for an echo signal with a maximum peak out of the echo signals acquired by the plurality of wave receivers, the tracking start point setting component sets the tracking start point at a signal waveform timing corresponding to the echo signal.

20. A waveform tracking method used in a waveform tracking device having a plurality of wave receivers disposed in a line to acquire echo signals of a transmitted ultrasound beam, the method comprising:
calculating a determination index that expresses a probability that the echo signal is a reflected wave from a measurement object;
setting a tracking start point for the echo signal for which the determination index satisfies a specific condition and that is received by a part of the plurality of wave receivers; and
setting tracking points for echo signals acquired from other wave receivers using the tracking start point as an origin.
